(19) United States
(12) Patent Application Publication
(10) Pub. No.: US 2002/0123479 A1
(43) Pub. Date: Sep. 5, 2002

(54) Immunostimulation mediated by gene-modified dendritic cells

(76) Inventors: Elizabeth S. Song, Howth (IE); Sunil Chada, Vista, CA (US); Virginia Lee, Cardiff, CA (US); Douglas J. Jolly, Leucadia, CA (US)

Correspondence Address: CHIRON CORPORATION, Intellectual Property, P.O. Box 8097, Emeryville, CA, 94662-8097, US

(21) Appl. No.: 09/976,576

(22) Filed: Oct. 12, 2001

Related U.S. Application Data
(63) Continuation of application No. 09/456,668, filed on Dec. 8, 1999, now abandoned.

(51) Int. Cl. A61K 48/00; A61K 39/395; C12N 7/01; C12N 15/867

(52) U.S. Cl. 514/44; 435/456; 424/93.21; 435/235.1; 424/144.1

(57) ABSTRACT

Compositions and methods useful for stimulating an immune response against one or more disease associated antigens by genetically modifying dendritic cells in vivo or ex vivo are provided. These compositions and methods allow for administration of lower dosages of gene delivery vehicles in order to achieve levels of immune stimulation comparable to those obtainable by conventional methods. Alternatively, administration of conventional dosages of gene delivery vehicles will enhance the resultant immune response.

### CLAIM TO PRIORITY

This application claims priority to two pending provisional applications filed Jan. 2, 1996 and Jan. 16, 1996. The priority applications were originally filed as utility applications and were assigned Ser. Nos. 08/581,867 and 08/587,285, respectively. The priority applications were converted to provisional applications on Nov. 28, 1996, which maintains the original filing dates for the provisional applications. As of the date of filing the instant application, the provisional applications serial numbers are not yet available from the U.S. Patent and Trademark Office. The priority applications are hereby incorporated by reference.

### TECHNICAL FIELD OF THE INVENTION

The present invention relates generally to recombinant DNA technology. In particular, the invention concerns compositions and methods useful for the prophylactic or therapeutic stimulation of the immune system of an animal by (i) in vivo transduction of dendritic cells or (ii) administration of dendritic cells transduced ex vivo with an expression vector functionally encoding at least one disease associated antigen.

### BACKGROUND OF THE INVENTION

Immune system stimulation to antigens associated with disease is an accepted approach to disease prevention. Traditional techniques have involved use of killed or attenuated-live vaccines made from various viral pathogens. The advent of recombinant DNA technology has enabled development of a new, more safe generation of vaccines to combat viral infections, wherein the immune stimulant is typically an immunogenic protein encoded by the pathogen.

Despite these technologies, few effective treatments or prophylactic measures have been developed for many viral diseases and cancer. Recently, several groups have reported immune induction in animals against HIV-encoded gene products through the use of gene therapy technology. Specifically, autologous fibroblasts transduced ex vivo with a retroviral vector encoding the HIV env/rev genes were injected into mice, non-human primates, and humans lead to induction of an immune response against these antigens. See Warner, et al., 1991; Laube, et al., 1993; and Zeigner, et al., 1994.

Such ex vivo approaches, however, are not practical for large scale vaccination programs, as a separate product, i.e., transduced autologous cells, must be generated for each patient. To overcome this and other shortcomings of ex vivo approaches, efforts are being undertaken to develop in vivo techniques wherein a gene delivery vehicle carrying an expression vector which directs expression of a disease-specific immunogen is administered directly to a patient. See WO 91/02805, WO 93/10814, WO 93/15207, WO 94/06921, WO 94/21792, and WO 95/07994. Irwin, et al. (1994) reported immune induction against specific immunogens following intramuscular injection of recombinant retroviruses in mice, rhesus monkeys, and baboons. Such approaches require no ex vivo manipulations, although problems such as low transduction efficiencies and low level expression of the desired immunogen remain to be solved. Accordingly, compositions and methods which enable improved immune stimulation against disease associated antigens are needed.

### SUMMARY OF THE INVENTION

It is the object of this invention to provide compositions and methods for the immunoprophylactic or immunotherapeutic treatment of animals, including mammals, particularly humans. The compositions of the invention can be used to deliver an immunogen, i.e., a disease associated antigen, to an animal in order to immunize it against disease, such as cancer or, alternatively, against bacterial, parasitic, or viral infections. Such immunization results from generation of a cell mediated immune response against the immunogen(s) (i) encoded by the expression vector delivered by a gene delivery vehicle which are then expressed and presented on the surface of an antigen presenting cell (APC) in vivo or (ii) presented on the surface of an APC, particularly a dendritic cell, transduced ex vivo by such an expression vector.

One aspect of the invention relates to gene delivery vehicles targeted to dendritic cells, be they in vivo or in vitro. Such gene delivery vehicles comprise a dendritic cell targeting element and an expression vector which directs expression of at least one disease associated antigen. In one embodiment, the expression vector is carried by a recombinant virus. Recombinant viruses useful in the practice of the invention include both DNA and RNA viruses. In preferred embodiments, the recombinant virus is one derived from either a negative strand RNA virus or a positive strand RNA virus. Representative positive strand RNA viruses from which recombinant viruses can be derived include retroviruses (e.g., avian leukosis virus, bovine leukemia virus, murine leukemia virus, mink-cell focus-inducing virus, murine sarcoma virus, reticuloendotheliosis virus, rous sarcoma virus, Mason-Pfizer monkey virus, baboon endogenous virus, endogenous feline retrovirus, gibbon ape leukemia virus, HIV I, HTLV I, and HTLV III), particularly murine retroviruses, togaviruses, e.g., alphaviruses, particularly Sindbis virus, Semliki Forest virus, and Venezuelan Equine Encephalitis virus, picornaviruses, and coronaviruses, with those derived from retroviruses and alphaviruses being preferred. Representative negative strand RNA viruses from which recombinant viruses can be derived include rhabdoviruses (e.g., vesicular stomatitis virus), myxoviruses, paramyxoviruses, orthomyxoviruses (e.g., influenza virus), and bunyaviruses. Useful DNA viruses from which recombinant viruses useful in practicing the invention may be derived include adenoviruses and adenoassociated viruses.

In other embodiments, the gene delivery vehicle is non-viral gene delivery vehicle, i.e., the expression vector is not carried by a virus. The expression vector will be either DNA or RNA, and can be linear or circularized. A particularly preferred expression vector is a eukaryotic layered vector initiation system. In one embodiment, the expression vector is complexed with one or more polynucleotide condensing agents. Polynucleotide condensing agents include polycations. Preferred polycations include polylysine, polyarginine, histones, protamines, spermidine, and spermine, with polylysine being particularly preferred. In another embodiment, the expression vector is complexed only with the dendritic cell targeting element. In yet another embodiment, the expression vector is associated with lipids, preferably being encapsulated in liposomes, particularly liposomes made of cationic lipids.

When a gene delivery vehicle is targeted to a dendritic cell, the dendritic cell targeting element can be any molecule which targets the gene delivery vehicle to a dendritic cell. Various embodiments include those wherein the dendritic cell targeting element is selected from the group consisting of a high affinity binding pair, an antibody reactive against a dendritic cell surface marker, and an antigen binding domain derived from an antibody reactive against a dendritic cell surface marker. Preferred high affinity binding pairs include those selected from the group consisting of biotin/avidin, cytostatin/papain and phosphonate/carboxypeptidase A. Preferred dendritic cell surface markers, against which antibodies (or antigen binding domains derived therefrom) can be generated to produce dendritic cell targeting elements, include CD 11c, CD 54, CD 58, CD 25, CD 11a, CD 23, CD 32, CD 40, CD 1, CD 45, MHC Class I, MHC Class II, Mac-1, Mac-2, and Mac-3. In another embodiment, the dendritic cell targeting element is a hybrid envelope protein, wherein the envelope portion is derived from a viral envelope protein, e.g., a retroviral envelope protein, and the dendritic cell targeting element is derived from a protein which specifically interacts with a molecule presented on a dendritic cell plasma membrane.

Expression vectors according to the invention direct expression of at least one disease associated antigen. Such antigens are preferably associated with diseases selected from the group consisting of cancer, a hyperproliferative disease, a bacterial infection, a parasitic infection, and a viral infection. The cancers that may be treated, inhibited, or prevented using the immunostimulatory compositions and methods described herein include breast cancer, colon cancer, melanoma, lung cancer, brain cancer, and leukemia, among others. Bacterial infections which can be treated, inhibited, or prevented include pneumonia, sepsis, tuberculosis, and staph infections, among others. Parasitic infections which can be treated include those which cause malaria (caused by protozoa of the genus Plasmodium, and include P. falciparum, P. malariae, P. ovale, and P. vivax), sleeping (caused by trypanosomes), and river blindness, among others. Viral infections which may be treated, inhibited, or prevented using the compositions and methods described herein include those caused by hepatitis A, hepatitis B, hepatitis C, non-A, non-B hepatitis, hepatitis delta agent, CMV, Epstein-Barr virus, HTLV I, HTLV II, and HIV I, among others.

In one embodiment, the expression vector codes for a single disease associated antigen. In other embodiments, the expression vector codes for multiple disease associated antigens, such as 2, 3, 4, 5, 6, 7, 8, 9, or 10 or more antigens. When multiple disease associated antigens are encoded by a single expression vector, the antigens may be associated with the same or different diseases. Alternatively, compositions comprising combinations of expression vectors, each encoding for one or more antigens associated with a disease different from those encoded by other expression vectors, may also be prepared. Compositions encoding for one or more antigens associated with different diseases are particularly useful as vaccines

In yet another embodiment of this aspect of the invention, the expression vector also encodes an immunomodulatory cofactor.

Preferred embodiments of the invention relate to pharmaceutical compositions comprising the various gene delivery vehicles of the invention and a pharmaceutically acceptable carrier or diluent. Particularly preferred embodiments include those wherein the pharmaceutical compositions are in solid form.

A second aspect of the invention concerns in vivo methods for producing genetically modified dendritic cells. Such methods comprise administering to an animal a gene delivery vehicle targeted to a dendritic cell, wherein the gene delivery vehicle comprises a dendritic cell targeting element and an expression vector which directs expression of at least one disease associated antigen.

In a related aspect, methods of prophylaxis are provided. In one embodiment, prophylaxis is achieved by administering to an animal a prophylactically effective amount of a gene delivery vehicle according to the invention. In another embodiment, prophylaxis is achieved by administering to an animal a prophylactically effective amount of a dendritic cell population transduced ex vivo with a gene delivery vehicle carrying an expression vector encoding genetic information sufficient to direct expression of a gene encoding at least an antigenic portion of a disease associated antigen.

In yet another related aspect of the invention, methods are provided for the therapeutic treatment, not prophylaxis, of disease. In one embodiment, therapeutic treatment is achieved by administering to an animal a therapeutically effective amount of a gene delivery vehicle targeted to a dendritic cell, wherein the gene delivery vehicle carries an expression vector encoding genetic information sufficient to direct expression of a gene encoding at least an antigenic portion of a disease associated antigen. In another embodiment, therapeutic treatment is achieved by administering to an animal a therapeutically effective amount of a dendritic cell population transduced ex vivo with a gene delivery vehicle carrying an expression vector encoding genetic information sufficient to direct expression of a gene encoding at least an antigenic portion of a disease associated antigen.

Such methods of prophylaxis or treatment of an animal can be accomplished by a single direct injection of gene delivery vehicles or a dendritic cell population transduced ex vivo at a single time point or multiple time points. In another embodiment, the method comprises administration of gene delivery vehicles of the invention or an ex vivo transduced dendritic cell population nearly simultaneously to multiple sites. Preferred routes of administration include the intravenous and subcutaneous routes. Preferred animals for prophylactic treatment by such methods include birds, mammals, and fish. Particularly preferred mammals include those selected from the group consisting of human, bovine, equine, canine, feline, porcine, and ovine animals.

Yet another aspect of the invention relates to ex vivo methods of producing genetically modified dendritic cells. In one embodiment, such methods comprise obtaining a population of cells comprised substantially of dendritic cells, i.e., greater than about 50% dendritic cells, more preferably greater than about 75% dendritic cells, more preferably still greater than about 90% dendritic cells, with greater than about 95% dendritic cells being particularly preferred, and genetically modifying that population of cells through introduction of a gene delivery vehicle according to the invention. In another embodiment, the method comprises obtaining a first population of cells containing dendritic cells, genetically modifying the first population of cells through introduction of a gene delivery vehicle according to the invention, and isolating a second population of cells comprised substantially of dendritic cells from the genetically modified first population of cells in part. Preferred methods for isolating cell populations comprised substantially of dendritic cells include affinity chromatography and FACS.

### BRIEF DESCRIPTION OF THE DRAWINGS

FIG. 1 illustrates some of the cell surface markers useful in identifying dendritic cells. Markers indicated with a (−) are not present on dendritic cells, and thus may be used in negative selection, strategies. Those marked (+) are present on the dendritic cell surface, with (++) and (+++) indicating particularly useful dendritic cell markers. The (+), (++), and (+++) dendritic cell markers also represent suitable targets to which gene delivery vehicles according to the invention can be targeted.

FIG. 2 is a flow chart depicting a splenocyte separation strategy using percoll density gradients.

FIG. 3 illustrates the effect of dendritic cell therapy on CT26.p24. Balb/c mice were injected with a total of 2×10⁵ tumor cells. On day 8, 15, and 22, animals were treated as in the figure. The tumor volume of the mice was measured twice a week.

FIG. 4 illustrates the effect of dendritic cell therapy on JC.p24. Balb/c mice were injected with a total of 3×10⁵ tumor cells. On day 8, 15, and 22, animals were treated as in the figure. The tumor volume of the mice was measured twice a week.

FIG. 5 shows the effect of dendritic cell immunization on JC.p24. Balb/c mice were immunized with splenic dendritic cells transduced with p24 twice (one week apart) followed by injection of 3×10⁵ tumor cells. The tumor volume of the mice was measured twice a week.

FIG. 6 shows the effect of dendritic cell immunization on JC.p24 following a single immunization of BM-DC.p24. Balb/c mice were immunized once with BM-DC.p24 One week later, they were injected with a total of 3×10⁵ tumor cells. The tumor volume of the mice was measured twice a week.

FIG. 7 shows the effect of dendritic cell immunization on JC p24. Balb/c mice were treated with D2SC/1.beta-gal and D2SC/1.p24 on days 1 and 8. They were subsequently injected with a total of 3×10⁵ tumor cells on day 15. The tumor volume of the mice was measured twice a week.

FIG. 8 illustrates the effect of dendritic cell immunization on CT26.p24. Balb/c mice were immunized on days 1 and 7 with the vaccines provided in the figure legend. On day 15, animals were injected with a total of 2×10⁵ tumor cells. The tumor volume of the mice was measured twice a week.

FIG. 9 shows comparisons between methods for antigen introduction to dendritic cells. This compares the retroviral vector vs protein or peptide loading. Balb/c mice were immunized on days 1 and 7 with the vaccines provided in the figure legend. On day 8, animals were injected with a total of 2×10⁵ CT26.beta-gal tumor cells. The tumor volume of the mice was measured twice a week.

### DEFINITION OF TERMS

The following terms are used throughout the specification. Unless otherwise indicated, these terms are defined as follows:

“Gene delivery vehicle” refers to a construct which is capable of delivering, and, within preferred embodiments expressing, one or more gene(s) or sequence(s) of interest in a host cell. Representative examples of such vehicles include viral vectors, naked DNA or RNA expression vectors, DNA or RNA expression vectors associated with cationic condensing agents, DNA or RNA expression vectors encapsulated in liposomes, and certain eukaryotic cells (e.g., producer cells). Within preferred embodiments of the invention, a gene delivery vehicle includes a targeting element, e.g., a member of a high affinity binding pair covalently linked to, expressed on, or included as an integral part of the exterior of the gene delivery vehicle.

“High Affinity Binding Pair” refers to a set a molecules which is capable of binding one another with a K_{D} of less than 10^{-y}M, wherein y is selected from the group consisting of 8, 9, 10, 11, 12, 13, 14 and 15. As utilized herein, the “K_{D}” refers to the disassociation constant of the reaction A+B=AB, wherein A and B are members of the high affinity binding pair. As understood in the art, as the affinity of the two molecules increases, K_{D} decreases. Affinity constants may be readily determined by a variety of techniques, including, for example, by Scatchard analysis (see Scatchard, Ann. N.Y. Acad. Sci. 51:660-672, 1949). Representative examples of suitable affinity binding pairs include biotin/avidin, cytostatin/papain and phosphonate/carboxypeptidase A.

“Targeting element” refers to a molecule which is capable of specifically binding a dendritic cell. Within the context of this invention, a targeting element is considered to specifically bind a dendritic cell when a biological effect is seen in that cell type after binding of the targeting element and its complement, or, when there is greater than a 10 fold difference, and preferably greater than a 25, 50 or 100 fold difference between the binding of the coupled targeting element to dendritic cells and non-target cells. Generally, it is preferable that the targeting element bind to dendritic cells with a K_{D} of less than 10⁻⁵M, preferably less than 10⁻⁶M, more preferably less than 10⁻⁷M, and most preferably less than 10⁻⁸M (as determined by Scatchard analysis, supra). In addition, when a high affinity binding pair is used for targeting, the targeting element will preferably bind dendritic cells with an affinity of at least 1 log (i.e., 10 times) less than the affinity constant of the high affinity binding pair. Suitable targeting elements are preferably non-immunogenic, not degraded by proteolysis, and not scavenged by the immune system. Particularly preferred targeting elements preferably have a half-life (in the absence of a clearing agent) in an animal of between 10 minutes and 1 week. Representative examples of suitable targeting elements are set forth below in more detail.

“Targeting dendritic cells” refers to methods that target dendritic cells. Such methods are defined as follows: the gene delivery vehicle possesses an element that causes increased transduction of dendritic cells following administration in any of a number of conventional ways, as compared to transduction that occurs when the gene delivery vehicle lacks the element; or the gene delivery vehicle is administered in a particular fashion or by a particular route so that transduction of dendritic cells is enhanced as compared to normal or conventional routes of administration.

“Clearing agent” refers to a molecule which binds and/or cross-links circulating, coupled targeting elements. Preferably, the clearing agent is non-immunogenic, specific to the coupled targeting element, and large enough to avoid rapid renal clearance. In addition, the clearing agent is preferably not degraded by proteolysis, and not scavenged by the immune system. Particularly preferred clearing agents include those which bind to the coupled targeting element at a site other than the affinity binding member, and most preferably, which bind in a manner that blocks the binding of the targeting element to its target. Numerous cleaving agents may be utilized within the context of the present invention, including for example those described by Marshall et al. in Brit. J. Cancer 69:502-507, 1994.

“Expression Vector” refers to a recombinant nucleic acid molecule (DNA or RNA) capable of directing expression of one or more genes encoding a disease associated antigen. The expression vector must include a promoter (unless the expression vector is designed for position-specific integration adjacent to a functional promoter) operably linked to the antigen-encoding gene(s), and a polyadenylation sequence. In certain embodiments, the expression vector is part of a plasmid construct. In addition to the expression vector components, the plasmid construct may also include one or more of the following: a bacterial origin of replication; one or more selectable markers; a signal which allows the plasmid construct to exist as single-stranded DNA (e.g., a M13 origin of replication); a multiple cloning site; and a “mammalian” origin of replication (e.g., a SV40 or adenovirus origin of replication). In other embodiments, the expression vector is a recombinant viral genome, and will be either RNA or DNA, depending on the particular viral system being utilized. Alternatively, the expression vector may comprise in vitro transcribed RNA. As used herein, “expression vector” also refers to a vector which, after introduction into a cell, is converted to a different form. For example, the RNA genome carried a recombinant retrovirus is reverse transcribed into DNA and integrated into the genome of the cell. For purposes of this invention, both RNA and DNA forms are “expression vectors.”

“Altered Cellular Component” refers to proteins and other cellular constituents which are either associated with rendering a cell tumorigenic, or are associated with tumorigenic cells in general but are not required or essential for rendering the cell tumorigenic. Before alteration, the cellular components man, be essential to normal cell growth and regulation, and include for example, proteins which regulate intracellular protein degradation, transcriptional regulation, cell-cycle control, and cell-cell interaction. After alteration, the cellular components no longer perform their usual regulatory functions, and hence the cell may experience uncontrolled growth. Representative examples of altered cellular components include ras*, p53*, Rb*, altered protein encoded by the Wilms' tumor gene, ubiquitin*, mucin*, protein encoded by the DCC, APC, and MCC genes, as well as receptors or receptor-like structures such as neu, thyroid hormone receptor, platelet derived growth factor (PDGF) receptor, insulin receptor, epidermal growth factor (EGF) receptor, and the colony stimulating factor (CSF) receptor. These as well as other cellular components are described in more detail below, as well as discussed in cited references.

“Non-tumorigenic” refers to altered cellular components which will not cause cellular transformation or induce tumor formation in nude mice. Representative assays which distinguish tumorigenic cellular components from non-tumorigenic cellular components are described in more detail below.

“Immunogenic” as utilized within the present invention refers to altered cellular components which are capable, under the appropriate conditions, of causing an immune response. This response must be cell-mediated and may include a humoral response. Representative assays which may be utilized to determine immunogenicity are described in more detail below.

Numerous aspects and advantages of the invention will be apparent to those skilled in the art upon consideration of the following detailed description which provides illumination of the practice of the invention.

### DETAILED DESCRIPTION OF THE INVENTION

The present invention is based on the discovery that expression and cell surface presentation of at least one disease associated antigen in dendritic cells can be used to generate a prophylactic or therapeutic immune response against the disease with which the antigen is associated. Moreover, it has been discovered that the efficiency of immune system stimulation mediated by genetically modified dendritic cells can be several orders of magnitude greater than that mediated by genetically modified fibroblasts, muscle, and other cell types. As a result, these discoveries enable improved approaches to gene therapy-mediated immune stimulation, either by reducing the dosage(s) required to achieve the desired prophylactic or therapeutic result or by enhancing immune responses.

An animal's ability to recognize and defend against foreign pathogens, i.e., to distinguish “self” from “non-self” (foreign), is essential to proper immune system function. The immune system comprises at least three components: a humoral (antibody) component; a complement component; and a cell-mediated component. The cell-mediated component comprises at least cytotoxic T-lymphocytes (CTLs), and also includes cells responsible for generating antibodies. CTLs are typically induced, or stimulated, by display of a cell surface recognition structure, such as a processed, pathogenic agent-specific peptide in conjunction with a MHC class I cell surface protein. Diseases suitable to treatment using an immunostimulation strategy include: viral infections, such as those caused by HBV (see WO 93/15207), HCV (see WO 93/15207), HPV (see WO 92/05248, WO 90/10459, EPO 133,123), Epstein-Barr Virus (see EPO 173,254; JP 1,128,788; and U.S. Pat. Nos. 4,939,088 and 5,173,414), Feline Leukemia Virus (see WO 93/09070, EPO 377,842, WO 90/08832, and WO 93/09238), Feline Immunodeficiency Virus (U.S. Pat. No. 5,037,753, WO 92/15684, WO 90/13573, and JP 4,126,085), HTLV I and II, and HIV (see WO 91/02805); cancers, such as melanoma, cervical carcinoma, colon carcinoma, renal carcinoma, breast cancer, ovarian cancer, prostate cancer, leukemias; and heart disease.

Dendritic cells are a system of antigen presenting cells that function to initiate immune responses (Steinman, R. (1991), Annu. Rev. Immunol., vol. 9:272-296; see also Research in Immunology, vol. 140, International Reviews in Immunology. vol. 6, Advances in Immunology, vol. 47, and Epidermal Langerhans Cells, ed. G. Schuler). Dendritic cells are found in many non-lymphoid tissues but can migrate via the afferent lymph or the blood stream to the T-dependent areas of lymphoid organs. In non-lymphoid organs, dendritic cells include Langerhans cells and interstitial dendritic cells. In the lymph and blood, they include afferent lymph veiled cells and blood dendritic cells, respectively. Dendritic cells are not found in the efferent lymph. In lymphoid organs, they include lymphoid dendritic cells and interdigitating cells. As used in the context of this invention, each of these cell types and their progenitors shall be referred to as “dendritic cells,” unless otherwise specified.

Dendritic cells have an unusual dendritic shape, are motile, and efficiently cluster and activate T cells that are specific for cell surface stimuli. Typically, dendritic cells in non-lymphoid organs, such as Langerhans cells and interstitial cells, become veiled cells (cells which continually extend and retract large lamellipodia) in the afferent lymph and blood which migrate to lymphoid tissues, where they can be isolated as dendritic or interdigitating cells.

Partially enriched populations of epidermal Langerhans cells, wherein Langerhans cells may comprise up to about 60% of the total cell population, may be readily prepared, since keratinocytes can be depleted from murine tissue using α-thy-1 (a monoclonal antibody) and complement plus adherence. Enriched preparations of human Langerhans cells can be prepared by substituting an anti-CD 1 antibody for α-thy-1. In culture, neither mouse nor human Langerhans cells are active antigen-presenting cells until after 1-3 days in culture, after which time they enlarge, express more MHC Class II and cell adhesion molecules, and lose Fc receptors, fully resembling blood and lymphoid dendritic cells. Cell populations containing more than 90% dendritic cells have been obtained from human blood, where, without enrichment, fewer than 0.1% of the white cells are dendritic cells. Such enrichment can be achieved by successive depletion of T cells, monocytes, and B plus NK cells to yield an initial population ranging from 30-60% dendritic cells. Greater purity is then obtained by panning or FACS using a monoclonal antibody, especially to CD45R_{A}, that selectively reacts to contaminants (Freudenthal, P., Steinman, R. M., Proc. Nat'l. Acad. Sci. (USA) (1990) 87:7698-7702.) To enrich for dendritic cells generally, selection for low buoyant density, non-adherence to plastic in culture (especially after one or more days), and absence of markers found on other cells is performed. Such methods deplete other cell types, but do not positively select dendritic cells.

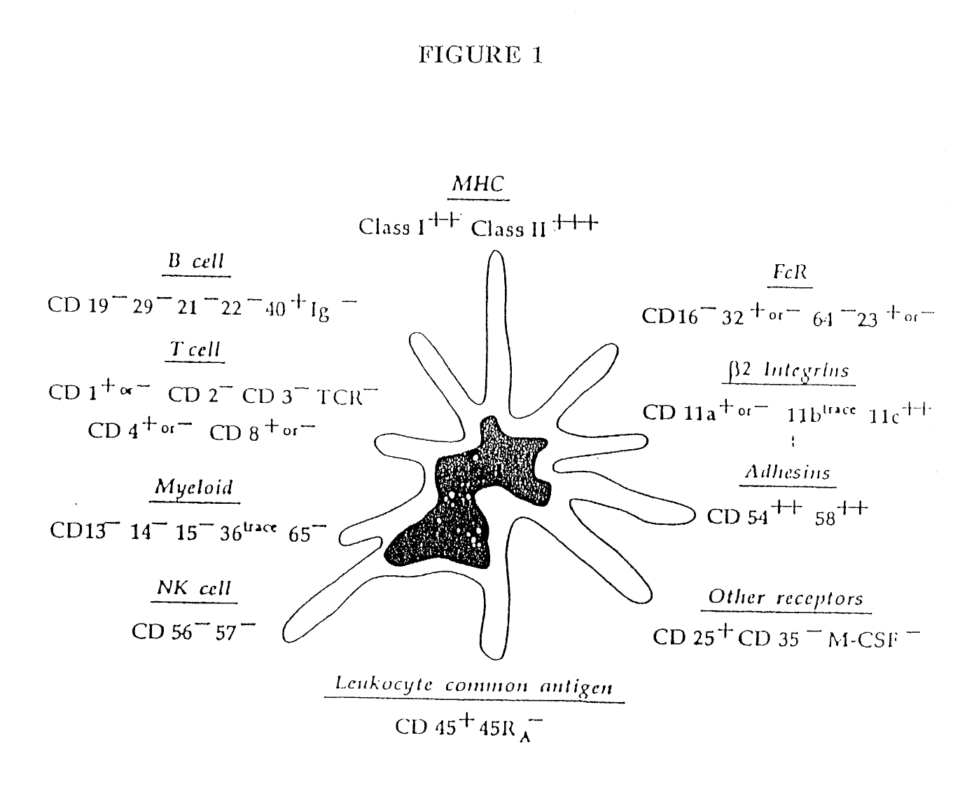

Dendritic cells express a distinct pattern of markers on their cell membranes. FIG. 1 illustrates this pattern by indicating the presence or absence of several distinct cell surface markers. Other markers which can be used to positively or negatively select for dendritic cells include ICAM-1 (CD 54), LFA-3 (CD 58), and CD 11b. Dendritic cells isolated from human or mouse blood, but not skin, express CD 11a or LFA-1. In skin, the immunostimulatory effect of dendritic cells may be enhanced by cytokines, particularly by GM-CSF.

Dendritic cells initiate T-dependent responses from quiescent lymphocytes. Once sensitized, T cells interact with other antigen presenting cells. Dendritic cell antigen processing activity is regulated. Only fresh cells, i.e., cells cultured for less than a day, isolated form skin or lymphoid organs present native proteins. After that time, they do not process antigens. In addition, dendritic cells are not actively phagocytic.

Gene therapy-mediated immunostimulation can be accomplished by various methods. For example, an expression vector encoding a disease-associated antigen or modified form thereof (collectively referred to hereinafter as “antigen”) can be delivered to dendritic cells to initiate an immune response against the antigen. Expression of the antigen may be transient or stable over time. Where an immune response is to be stimulated by an antigen from a pathogenic agent, e.g., a virus, a bacteria, or neoplastic or otherwise diseased autologous cell, the expression vector preferably encodes a modified form of the antigen which has reduced pathogenicity relative to the native antigen but still stimulates an immune response thereto.

In the particular case of disease caused by viral infection (e.g., AIDS caused by HIV). the immunity stimulating expression product encoded by the expression vector is of a form which will elicit either or both an HLA Class I- or Class II-restricted immune response. For HIV, a preferred antigen for immunostimulation is derived from the envelope protein, preferably selected from gp 160, gp 120, and gp 41, which has been modified to reduce pathogenicity, in particular, to reduce the possibility of syncytia, to avoid expression of epitopes leading to a disease enhancing immune response, to remove immunodominant but strain-specific epitopes, or to present several strain-specific epitopes. Other HIV genes or combinations of genes which may be expressed for this purpose include gag, pol, rev, vif, nef, prot, gag/pol, gag prot, etc. Additionally, immunogenic portions from other desired antigens may be expressed. Immunogenic portion(s) of desired antigens may be of varying length, preferably at least 9 amino acids and may include the entire protein. As those in the art will appreciate, this and similar immunostimulatory approaches can be employed in the treatment of numerous diseases.

In further embodiments, expression vectors direct expression of at least one gene of interest which encode one or more immunogenic portions of disease-associated antigens. As used herein, a gene of interest codes for at least one product capable of immunostimulation. A “disease-associated” antigen is one associated with rendering a cell (or organism) diseased, or with the disease state in general but which is not required for rendering the cell diseased. A wide variety of “disease-associated” antigens are known, including immunogenic, non-tumorigenic altered cellular components which are normally associated with tumor cells (see U.S. Ser. No. 08/104,424 and WO 93/10814). Representative examples of altered cellular components associated with tumor cells include ras* (wherein “*” refers to antigens altered to be non-tumorigenic), p53* (see Levine et al., Nature 351:453, 1991), Rb* (Bookstein et al., Science 247:712, 1990); altered protein encoded by Wilm's tumor gene (Call et al., Cell 60:509,1990); ubiquitin* (Mafune et al., Arch.-Surg. 126:462, 1991); mucin (Jerome et al., Cancer Res. 51:2908, 1991); and proteins encoded by the DCC (deleted in colorectal carcinomas; Fearon et al., Science 247:49, 1990, and Solomon, Nature 343:412, 1990) MCC (mutated in colorectal carcinomas; Kinzler et al., Science 251:1366, 1991, and Nishiho et al., Science 253:665, 1991), and APC genes. Tumor associated antigens that correspond to a cell mediated immune response, such as MAGE-1, MAGE-3, MART-1, and gp100, are also included among “disease associated antigens” as used herein. “Disease-associated” antigens should also be understood to include all or portions of various eukaryotic, prokaryotic or viral pathogens. See co-owned U.S. Ser. No. 07/800,328 and U.S. Ser. No. 08/104,424 for additional details.

Nucleic acid molecules that encode the above-described products, as well as other nucleic acid molecules that are advantageous for use within the present invention, may be readily obtained from a variety of sources, including, for example, depositories such as the American Type Culture Collection, or from commercial sources such as British Bio-Technology Limited (Cowley, Oxford England). Alternatively, cDNA sequences for use with the present invention may be obtained from cells which express or contain the sequences, such as by RT PCR from isolated mRNA. Nucleic acid molecules suitable for use with the present invention may also be synthesized in whole or in part, for example, on an Applied Biosystems Inc. DNA synthesizer (e.g., ABI DNA synthesizer model 392 (Foster City, Calif.).

A. Gene Delivery Vehicles

A gene delivery vehicle (“GDV”) is a composition capable of delivering a nucleic acid molecule, specifically an expression vector, to a eukaryotic cell. Representative examples of gene delivery vehicles include recombinant viral vectors (e.g., retroviruses; see WO 89/09271, and alphaviruses such as Sindbis; see WO 95/07994), other recombinant and non-recombinant viral systems (e.g., adenovirus; see WO 93/19191), nucleic acid molecules associated with one or more condensing agents (see WO 93/03709), nucleic acid molecules associated with liposomes (Wang, et al., PNAS 84:7851, 1987), modified bacteriophage, or bacteria. In whatever form, the GDV carries an expression vector which directs expression of at least one disease associated antigen in a target cell.

In one embodiment, the GDV is a recombinant virus derived from a virus such as an astrovirus, coronavirus, orthomyxovirus, papovavirus, paramyxovirus, parvovirus, picomavirus, poxvirus, retrovirus, togavirus or adenovirus. In a preferred embodiment, the recombinant viral vector is a recombinant retroviral vector. Retroviral GDVs may be readily constructed from a wide variety of retroviruses, including for example, B, C, and D type retroviruses, as well as spumaviruses and lentiviruses (see RNA Tumor Viruses, Second Edition, Cold Spring Harbor Laboratory, 1985). Such retroviruses may be readily obtained from depositories or collections such as the American Type Culture Collection (ATCC, Rockville, Md.), or isolated from known sources using commonly available techniques. Numerous retroviral GDVs which may be utilized in practicing the present invention are described in U.S. Pat. Nos. 5,219,740 and 4,777,127, EP 345,242 and WO 91/02805.

Particularly preferred recombinant retroviruses are derived from retroviruses which include avian leukosis virus (ATCC Nos. VR-535 and VR-247), bovine leukemia virus (VR-1315), murine leukemia virus (MLV), mink-cell focus-inducing virus (Koch et al., J. Vir. 49:828, 1984; and Oliff et al., J. Vir. 48:542, 1983), murine sarcoma virus (ATCC Nos. VR-844, 45010 and 45016), reticuloendotheliosis virus (ATCC Nos VR-994, VR-770 and 45011), rous sarcoma virus, Mason-Pfizer monkey virus, baboon endogenous virus, endogenous feline retrovirus (e.g., RD114), gibbon ape leukemia virus (GALV), human immunodeficiency virus (HIV), HTLV I, HTLV III, and mouse or rat gL30 sequences used as a retroviral vector. Particularly preferred strains of MLV from which recombinant retroviruses can be generated include 4070A and 1504A (Hartley and Rowe, J. Vir. 19:19, 1976), Abelson (ATCC No. VR-999), Friend (ATCC No. VR-245). Graffi (Ru et al., J. Vir. 67:4722, 1993; and Yantchev Neoplasma 26:397, 1979), Gross (ATCC No. VR-590), Kirsten (Albino et al., J. Exp. Med. 164:1710, 1986), Harvey sarcoma virus (Manly et al., J. Vir. 62:3540, 1988; and Albino et al., J. Exp. Med. 164:1710, 1986) and Rauscher (ATCC No. VR-998), and Moloney MLV (ATCC No. VR-190). A particularly preferred non-mouse retrovirus is rous sarcoma virus. Preferred rous sarcoma viruses include Bratislava (Manly et al., J. Vir. 62:3540, 1988; and Albino et al., J. Exp. Med. 164:1710, 1986), Bryan high titer (e.g., ATCC Nos. VR-334, VR-657, VR-726, VR-659, and VR-728), Bryan standard (ATCC No. VR- 140), Carr-Zilber (Adgighitov et al., Neoplasma 27:159, 1980), Engelbreth-Holm (Laurent et al., Biochem Biophys Acta 908:241, 1987), Harris, Prague (e.g., ATCC Nos. VR-772, and 45033), and Schmidt-Ruppin (e.g. ATCC Nos. VR-724, VR-725, VR-354).

Any of the above retroviruses may be readily utilized in order to assemble or construct retroviral GDVs given the disclosure provided herein and standard recombinant techniques. In addition, portions of the retroviral GDVs may be derived from different retroviruses. For example, recombinant retrovirus may comprises LTRs from a murine sarcoma virus, a tRNA binding site from a rous sarcoma virus, a packaging signal from a MLV, and an origin of second strand synthesis from an avian leukosis virus. These recombinant retroviral vectors may be used to generate transduction competent retroviral vector particles by introducing them into appropriate packaging cell lines (see U.S. Ser. No. 07/800,921, filed Nov. 29, 1991). In addition, recombinant retroviruses can be produced which direct the site-specific integration of the recombinant retroviral genome into specific regions of the host cell DNA. Such site-specific integration can be mediated by a chimeric integrase incorporated into the retroviral particle. See, for example, U.S. Ser. No. 08/445,466 filed May 22, 1995. It is preferable that the recombinant viral vector is a replication defective recombinant virus.

Preferably, a recombinant retrovirus based expression vector should include a 5′LTR, a tRNA binding site, a packaging signal, a nucleic acid molecule encoding one or more genes of interest (i.e., genes encoding at least one disease associated antigen), an origin of second strand DNA synthesis, and a 3′LTR. Such expression vectors may also include a transcriptional promoter/enhancer or locus defining element(s). or other elements which control gene expression by means such as alternate splicing, nuclear RNA export, post-translational modification of messenger, or post-transcriptional modification of protein. Optionally, the vector may also include one or more selectable markers that confer resistance of vector transduced or transfected cells to hygromycin, phleomycin, histidinol, or methotrexate, as well as one or more specific restriction sites and a translation termination sequence. Alternatively, the vector may encode a marker, e.g., thymidine kinase (TK), which confers sensitivity so that vector-modified cells can later be eliminated.

Within another embodiment, GDVs are derived from togaviruses. Preferred togaviruses include alphaviruses, in particular, those described in co-owned U.S. Ser. No. 08/405,627 and WO 95/07994. A representative alphavirus in Sindbis virus. Briefly, recombinant Sindbis expression vectors typically comprise a 5′ sequence capable of initiating Sindbis virus transcription, a nucleotide sequence encoding Sindbis non-structural proteins, a modified or inactivated viral junction region, a Sindbis RNA polymerase recognition sequence, at least one gene of interest, and a polyadenylate tract. Corresponding regions from other alphaviruses may be used in place of those described above.

In a preferred embodiment, a recombinant alphaviral vector does not encode structural proteins and the gene(s) of interest are located downstream from the viral junction region. In vectors having a second viral junction region, the gene(s) of interest may be located downstream from the second viral junction region. In such instances, the vector may further comprise a polylinker located between the viral junction region and the gene(s) of interest.

Other recombinant togaviral vectors that may be utilized in the present invention include those derived from Semliki Forest virus (ATCC VR-67; ATCC VR-1247), Middleberg virus (ATCC VR-370). Ross River virus (ATCC VR-373; ATCC VR-1246), Venezuelan equine encephalitis virus (ATCC VR-923; ATCC VR-1250; ATCC VR-1249; ATCC VR-532), and those described within U.S. Pat. Nos. 5,091,309, 5,217,879, and WO 92/10578. The above described recombinant Sindbis expression vector, as well as numerous similar vector constructs, may be readily prepared essentially as described in U.S. Ser. No. 08/405,627.

In another embodiment, the recombinant viral vector is a recombinant adenoviral vector. Such expression vectors may be readily prepared and utilized given the disclosure provided herein (see Berkner, Biotechniques 6:616, 1988, and Rosenfeld et al., Science 252:431, 1991, WO 93/07283, WO 93/06223, and WO 93/07282).

Other viral vectors suitable for use in the present invention include, for example, those derived from poliovirus (Evans et al., Nature 339:385, 1989, and Sabin et al., J. Biol. Standardization 1:115, 1973) (ATCC VR-58); rhinovirus (Arnold et al., J. Cell. Biochem. L401, 1990) (ATCC VR-1110); pox viruses, such as canary pox virus or vaccinia virus (Fisher-Hoch et al., PNAS 86:317, 1989; Flexner et al., Ann. N.Y. Acad. Sci. 569:86, 1989; Flexner et al., Vaccine 8:17, 1990; U.S. Pat. No. 4,603,112 and U.S. Pat. No. 4,769,330; WO 89/01973) (ATCC VR-111; ATCC VR-2010); SV40 (Mulligan et al., Nature 277:108, 1979) (ATCC VR-305), (Madzak et al., J. Gen. Vir. 73:1533, 1992); influenza virus (Luytjes et al., Cell 59:1107, 1989; McMicheal et al., The New England Journal of Medicine 309:13, 1983; and Yap et al., Nature 273:238, 1978) (ATCC VR-797); parvovirus such as adeno-associated virus (Samulski et al., J. Vir. 63:3822, 1989, and Mendelson et al., Virology 166:154, 1988) (ATCC VR-645): herpes simplex virus (Kit et al., Adv. Exp. Med. Biol. 215:219, 1989) (ATCC VR-977; ATCC VR-260); Nature 277: 108, 1979); human immunodeficiency virus (EPO 386,882, Buchschacher et al., J. Vir. 66:2731, 1992); measles virus (EPO 440,219) (ATCC VR-24); A (ATCC VR-67; ATCC VR-1247), Aura (ATCC VR-368), Bebaru virus (ATCC VR-600; ATCC VR-1240), Cabassou (ATCC VR-922), Chikungunya virus (ATCC VR-64; ATCC VR-1241), Fort Morgan (ATCC VR-924), Getah virus (ATCC VR-369; ATCC VR-1243), Kyzylagach (ATCC VR-927), Mayaro (ATCC VR-66), Mucambo virus (ATCC VR-580; ATCC VR-1244), Ndumu (ATCC VR-371), Pixuna virus (ATCC VR-372; ATCC VR-1245), Tonate (ATCC VR-925), Triniti (ATCC VR-469), Una (ATCC VR-374), Whataroa (ATCC VR-926), Y-62-33 (ATCC VR-375), O'Nyong virus, Eastern encephalitis virus (ATCC VR-65; ATCC VR-1242), Western encephalitis virus (ATCC VR-70; ATCC VR-1251; ATCC VR-622; ATCC VR-1252), and coronavirus (Hamre et al., Proc. Soc. Exp. Biol. Med. 121:190, 1966) (ATCC VR-740).

In another embodiment of the invention, the GDV comprises a DNA or RNA expression vector. Representative examples of such GDVs include plasmids, cosmids, and linear double and single stranded polynucleotides. Such vectors should include the genetic information required for expression of the disease associated antigen(s) in dendritic cells, i.e., a promoter, an open reading frame encoding the disease associated antigen, a transcription termination signal, and a polyadenylation signal. One advantage of such polynucleotide systems is the ease with which large quantities of a given gene delivery vehicle can be produced. Large amounts of RNA may be produced by in vitro transcription using any of a variety of techniques known in the art. Similarly, large amounts of DNA can readily be cultured in bacteria.

In a related embodiment, the GDV comprises a DNA or RNA expression vector associated with a condensing agent (e.g., polycations). Polycations condense the expression vector by masking the negatively charged phosphate backbone, permitting the molecule to fold into a more compact form. Polycations useful in this embodiment of the invention include, among others, polylysine, polyarginine, histones, protamines, spermidine, spermine, and other highly basic proteins or polypeptides. Such polycations may be modified to incorporate targeting elements and/or to decrease immunogenicity of nucleic acid/polycation complexes. For example, the polycation can be chemically conjugated to one or more polyalkylene glycols, e.g., polyethylene glycol. Alternatively (or additionally), the polycation can be conjugated with one or more polysaccharides. Briefly, chemical conjugation involves forming a covalent linkage between the polycation and the polyalkylene or polysaccharide. Many suitable methods for making such linkages are known in the art, for instance, see co-owned U.S. Ser. No. 08/366,787. In general, the polyalkylene or polysaccharide is prepared for conjugation by creating an active group in place of a terminal hydroxyl group. A preferred active group for polyalkylene glycols is N-hydroxysuccinimide. A preferred reactive group for polysaccharides is an aldehyde, which is present on many natural sugars or which may be generated by chemical oxidation. It will be recognized, however, that other reactive groups may be prepared by well-known chemical methods for use in the practice of this invention.

Alternatively, the compound to be conjugated may be a synthetic polymer, other than PEG. Suitable polymers include homopolymers and copolymers which have either terminal or pendant functional group which is or may be converted to a reactive functional group for coupling to a polycation. A polymer's pendant functional groups result from a functional group present in the monomer from which the polymer is derived. Alternatively, polysaccharides may be conjugated to polycations to reduce their immunogenicity (see U.S. Ser. No. 08/366,787, supra). Polysaccharides suitable for conjugation to a polycation include homo- or hetero-polymers of D-glucose, D-mannose, D-galactose, L-galactose, D-xylose, D-arabinose, D-glucosamine, D-glucoronic acid, N-acetyl-muramic acid and N-acetyl neuraminic acid. Natural polysaccharides that may be used include dextran, alpha-amylose, amylopectin, amylase-modified polysaccharides, fructans, such as mannans, xylans, and arabinans. Derivatives of these polysaccharides, such as carboxy methyl cellulose, may also be used. Dextran is a preferred polysaccharide because of its widespread use as a plasma substitute in current clinical practice.

In an alternative embodiment, the GDV is a DNA or RNA expression vector associated with a liposome. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome to release its contents into the cytoplasm. Prior to interaction with the surface of a cell, however, the liposome membrane acts as a relatively impermeable barrier which sequesters and protects its contents, for example, from degradative enzymes in the plasma. Additionally, because a liposome is a synthetic structure, specially designed liposomes can be produced that incorporate desirable features (see Stryer, L., Biochemistry, pp:236-240, 1975 (W. H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PNAS 84: 7851, 1987, Plant et al., Anal. Biochem. 176:420, 1989, and U.S. Pat. No. 4,762,915). Liposomes can encapsulate a variety of nucleic acid molecules including DNA, RNA, plasmids, and other expression vectors useful in the practice of the present invention.

In yet another embodiment, the GDV is a modified bacteriophage. The expression vector carried by such GDVs may be engineered to contain a wide variety of immunity stimulating genes under the control of various promoters and other regulatory elements. In addition, expression vectors carried by such GDVs may be modified to eliminate most or nearly all bacteriophage sequences, thereby greatly increasing therapeutic gene(s) packaging capability. A representative bacteriophage GDV (based on bacteriophage lambda) is described in co-owned U.S. Ser. No. 08/366,522. In one embodiment, the only lambda nucleotide sequences contained in the expression vector are the cos sites at the 5′ and 3′ ends of the linear DNA to be packaged, leaving up to about 50 kb available for gene(s) of interest or other sequences. These and other cosmid versions of such GDVs require use of specific mutant gpJ-containing in vitro packaging extracts to generate infectious bacteriophage particles. Also included in the expression vector is an origin of replication (e.g., Co1E1) which allows replication in bacteria, and frequently a gene coding for a selectable marker. The nucleic acid molecules are cloned into the cosmid vector between the cos sites (see U.S. Ser. No. 08/366,522, filed Dec. 30, 1994).

In another embodiment, the GDV is a bacterial cell comprising a nucleic acid molecule for delivery to eukaryotic cells. For example, the bacterial cell may express and present a cytotoxic agent, such as an anti-tumor agent, on its surface or, alternatively, secrete it into the surrounding medium. Representative examples of bacterial cell GDVs include BCG (Stover, Nature 351:456, 1991) and Salmonella (Newton et al., Science 244:70, 1989).

In the context of protein expression, the expression vector carried by the GDV may include promoter elements (e.g., a promoter for RNA polymerase II or an RNA replicase, an event-specific promoter, and a tissue-specific promoter), a signal that directs polyadenylation, a selectable marker (e.g., thymidine kinase or a marker which confers resistance to a compound such as hygromycin, neomycin, phleomycin, histidinol, or methotrexate), as well as one or more restriction sites and a translation termination sequence. If the GDV is a retroviral particle, the GDV must include a retroviral packaging signal and long terminal repeat (LTR) sequences appropriate to the retrovirus used.

In some embodiments, the expression vector may encode more than one gene of interest. Expression of multiple genes of interest may be controlled either by a single promoter, or preferably, by additional secondary promoters or other elements (e.g., an internal ribosome binding site or “IRBS”). For example, an expression vector may, in addition to directing expression of at least one disease associated antigen, direct the expression of a molecule such as interleukin-12 (IL-12), interleukin-15 (IL-15), IL-2, g-interferon (g-IFN), or another molecule which acts to increase cell-mediated presentation in the T_{H}1 pathway, in order to enhance immune presentation and processing of the disease-associated antigen.

In addition to the GDVs described above, a targeted GDV according to the invention may carry a eukaryotic layered vector initiation system or other expression systems. Eukaryotic layered vector initiation systems are DNA expression vectors derived from positive strand RNA viruses and are capable of directing the synthesis of viral RNA in vivo. Such system comprise a 5′ promoter, a construct capable of (i) expressing one or more gene(s) of interest and (ii) replicating in a cell either autonomously or in response to one or more factors, a polyadenylation sequence, and a transcription termination sequence. Briefly, eukaryotic layered vector initiation systems provide a two stage or “layered” mechanism which controls expression of genes encoded thereby. The first “layer” initiates transcription of the second “layer” and comprises a promoter which is capable of initiating the 5′ synthesis of RNA from cDNA (e.g., a 5′ promoter). a 3′ transcription termination site, as well as one or more splice sites and/or a polyadenylation site, if desired. Representative promoters include both eukaryotic (e.g., pol I, II, or III) and prokaryotic promoters, and inducible or non-inducible (i.e., constitutive) promoters, such as, for example. Murine Leukemia virus promoters (e.g., MoMLV), metallothionein promoters, the glucocorticoid promoter. Drosophila actin 5C distal promoter, SV 40 promoter, heat shock protein 65 promoter, heat shock protein 70 promoter, immunoglubulin promoters, Mouse polyoma virus promoter (“Py”), rous sarcoma virus (“RSV”), BK virus and JC virus promoters. MMTV promoter, alphavirus junction region, CMV promoter, Adenovirus VA1RNA, rRNA promoter, tRNA methionine promoter, CaMV 35S promoter, nopaline synthetase promoter, and the lac promoter. Suitable replicases include those encoded by the non-structural genes of alphaviruses (such as Sindbis or Semliki Forest virus) and other strong RNA polymerases such as those from bacteriophages (e.g., T7 or SP6 RNA polymerase). The second layer replicates in a cell either autonomously or in response to one or more factors and comprises one or more genes of interest. See co-owned U.S. Ser. No. 08/404,796 for additional details in the construction of such systems.

GDVs, including GDVs coupled to one or more distinct dendritic cell targeting element types, can be further modified. One particularly useful modification includes use of compounds conjugated to polycations and gene delivery vehicles that reduce immunogenicity of the resulting compositions. See co-owned U.S. Ser. No. 08/366,787.

B. Genes of Interest

GDVs useful in the practice of this invention above include or contain one or more genes of interest, i.e., nucleic acid molecules which encode at least one antigen associated with or characteristic of the disease to be treated or prevented. Such antigens include those from altered cellular components and antigens from foreign organisms or other pathogens.

In one embodiment, expression vectors are provided which direct expression of an immunogenic, non-tumorigenic, altered cellular component. One such altered cellular component is a non-tumorigenic, altered ras (ras*). The ras* gene is causally linked to the neoplastic phenotype, and indeed may be necessary for the induction and maintenance of tumorigenesis, in a wide variety of distinct cancers, such as pancreatic carcinoma, colon carcinoma and lung adenocarcinoma. In addition, ras* genes are found in pre-neoplastic tumors and, therefore, immunostimulation may be used as a prophylactic vaccine prior to detection of a malignant tumor.

Mutation of the ras gene is believed to be an early event in carcinogenesis (Kumar et al., Science 248:1101-1104, 1990) which, if treated early, may prevent tumorigenesis. The spectrum of mutations occurring in the ras* genes found in a variety of cancers is quite limited. Tumorigenic mutations in ras* occur primarily (in vivo) in only 3 codons: 12; 13: and 61. Codon 12 mutations are the most prevalent in both human and animal tumors.

Table 1 below summarizes known in vivo mutations (codons 12, 13 and 61) which activate human ras, as well as potential mutations which have in vitro transforming activity. Potential mutations with in vitro transforming activity were produced by the systematic substitution of amino acids for the normal codon (e.g., other amino acids were substituted for the normal glycine at position 12).

TABLE 1
Amino Acid Substitutions that Activate Human ras Proteins

| | | | | | | | | | |
|---|---|---|---|---|---|---|---|---|---|
| Amino | Gly | Gly | Ala | Gln | Glu | Asn | Lys | Asp | |
| Acid | | | | | | | | | |
| Mutant | 12 | 13 | 59 | 61 | 63 | 116 | 117 | 119 | |
| Codon | | | | | | | | | |
| In vivo | | Val | Asp | | Arg | | | | |
| | Arg | Val | | His | | | | | |
| | Asp | Arg | | Leu | | | | | |
| | Cys | | | | | | | | |
| | Ala | | | | | | | | |
| | Ser | | | | | | | | |
| | Phe | | | | | | | | |
| In vitro | | Ala | Ser | Thr | Val | Lys | His | Glu | His |
| | Asn | | | Ala | | Ile | Arg | Glu | |
| | Gln | | | Cys | | | | Ala | |
| | Glu | | | Asn | | | | Asn | |
| | His | | | Ile | | | | | |
| | Ile | | | Met | | | | | |
| | Leu | | | Thr | | | | | |
| | Lys | | | Tyr | | | | | |
| | Met | | | Trp | | | | | |
| | Phe | | | Phe | | | | | |
| | Ser | | | Gly | | | | | |
| | Thr | | | | | | | | |
| | Trp | | | | | | | | |
| | Tyr | | | | | | | | |

Mutations such as those listed above result in proteins containing different primary amino acid sequences. As a result, an immune response directed against these regions may be utilized to destroy tumorigenic cells containing the altered sequences (ras*).

Another gene of interest which may be encoded by an expression vector according to the invention codes for an altered p53 (p53*) gene. p53 is a nuclear phosphoprotein initially classified as an oncogene gene product (Linzer and Levine, Cell 17:43-52, 1979; Lane and Crawford, Nature 278:261-263, 1979). It was later discovered that the original p53 cDNA clones were mutant forms of p53 (Hinds et al., J. Virol. 63:739-746, 1989) and that p53 is a tumor suppressor gene which negatively regulates the cell cycle. Mutation of this gene may lead to tumor formation. Of colon carcinomas that have been studied. 75%-80% show a loss of both p53 alleles through deletion or point mutation. Similar mutations are found in lung cancer, and in brain and breast tumors.

The majority of p53 mutations (e.g., p53*¹, p53*², etc.) are clustered between amino acid residues 130 to 290 (see co-owned U.S. Ser. No. 08/104,424). Four regions of the gene that are particularly affected occur at residues 132-145, 171-179, 239-248, and 272-286. Three “hot spots” within these regions that are of particular interest occur at residues 175, 248 and 273 (Levine et al., Nature 351:453-456, 1991). These alterations, as well as others which are described above, result in the production of protein(s) which contain mutant amino acid sequences.

Once a sequence encoding the altered cellular component has been obtained, it is necessary to ensure that the sequence encodes a non-tumorigenic protein in order for it to be used prophylactically or therapeutically. Various assays to assess tumorigenicity of a particular cellular component are known (see U.S. Ser. No. 08/104,424). Tumor formation in nude mice or rats is a particularly sensitive method for determining the tumorigenicity of a particular cellular component. Nude mice lack a functional cellular immune system (i.e., do not possess CTLs), and therefore provide a useful in vivo model in which to test the tumorigenic potential of cells. Non-tumorigenic cells do not display uncontrolled growth properties when introduced into nude mice. In contrast, transformed cells rapidly proliferate and generate tumors. Tumorigenicity may also be assessed by visualizing colony formation in soft agar (Macpherson and Montagnier, Vir. 23:291-294, 1964). Transgenic animals, such as transgenic mice, may also be utilized to assess the tumorigenicity of an altered cellular component. (Stewart et al., Cell 38:627-637, 1984; Quaife et al., Cell 48:1023-1034, 1987; and Koike et al., Proc. Natl. Acad. Sci. USA 86:5615-5619, 1989.) Dysregulated expression of the transgene may serve as a model for the tumorigenic potential of the newly introduced genes.

If the altered cellular component is associated with making the cell tumorigenic, the altered cellular component must be made non-tumorigenic. For example, the gene of interest which encodes the altered cellular component may be truncated to render the gene product non-tumorigenic, although any truncation must leave intact at least one immunogenic amino acid sequence. Alternatively, multiple translational termination codons may be introduced downstream of the that portion of the gene encoding an immunogenic region. Insertion of termination codons prematurely terminates protein translation, thus preventing expression of the transforming portion of the protein.

In one embodiment, the ras* gene is truncated in order to render the ras* protein non-tumorigenic. Briefly, the carboxy-terminal amino acids of ras* functionally allow the protein to attach to the cell membrane. Truncation of these sequences renders the altered cellular component non-tumorigenic. Preferably, the ras* gene is truncated in the purine ring binding site, for-example around the sequence which encodes amino acid number 110. The ras* gene sequence may be truncated such that as few as about 20 amino acids (including the altered amino acid(s)) are encoded by the expression vector, although preferably, as many amino acids, as possible should be expressed (while maintaining non-tumorigenicity).

In another embodiment, the p53* protein is truncated to render the cellular component non-tumorigenic. In a preferred embodiment, p53* is truncated to a sequence which encodes all four “hot spots.”

Other altered cellular components which are oncogenic may also be truncated in order to render them non-tumorigenic. For example, both neu and bcr/abl may be truncated to render them non-tumorigenic. Non-tumorigenicity may be confirmed by assaying the truncated altered cellular component as described above.

It should be noted, however, that if the altered cellular component is only associated with non-tumorigenic cells in general, and is not required or essential for making the cell tumorigenic, then it is not necessary to render the cellular component non-tumorigenic. Representative examples of such altered cellular components which are not tumorigenic include Rb*, ubiquitin*, mucin*, and any tumor associated antigen that correspond to a cell mediated immune response, such as MAGE-1, MAGE-3, MART-1, and gp100.

As discussed above, the altered cellular component must also be immunogenic to generate an appropriate immune response. Immunogenicity is preferably determined by an immunogenicity assay. Representative assays include an ELISA, which detects the presence of antibodies against the newly introduced vector, as well as assays which test for T helper cells such as gamma-interferon assays, IL-2 production assays, and proliferation assays.

In another embodiment of the present invention, more than one altered cellular component is co-expressed from the same expression vector. It will be evident to one of ordinary skill in the art that a variety of combinations can be made. Preferred combinations include expression vectors encoding altered two or more genes selected from the group consisting of ras, p53. DCC APC and MCC genes to treat or prevent colon cancer. Analogous methodology can be utilized to treat other cancers. For example, an expression vector which coexpresses mucin*, ras*, neu, BRCA1* and p53* can be utilized to treat breast cancer.

In other embodiments of the invention, expression vectors are provided which direct expression of immunogenic portions of antigens from foreign organisms or other pathogens. These antigens are also referred to herein as “disease associated antigens.” Representative examples of such antigens include antigens from bacteria (e.g., E. coli, streptococcus, staphylococcus, mycobacteria, etc.). fungi, parasites, and viruses (e.g., influenza virus, HIV, and Hepatitis A, B and C Virus (“HAV”, “HBV” and “HCV”, respectively), human papiloma virus (“HPV”), Epstein-Barr Virus (“EBV”), herpes simplex virus (“HSV”), hantavirus, HTLV I, HTLV II, cytomegalovirus (“CMV”), and feline leukemia virus). As utilized herein, “immunogenic portion” refers to a portion of the respective antigen which is capable, under the appropriate conditions, of causing an immune response (i.e., cell-mediated or humoral). “Portions” may be of variable size, but are preferably at least 9 amino acids long, and may include the entire antigen.

In one embodiment, an expression vector is provided which directs expression of one or more immunogenic portions of a HBV antigen. The HBV genome is has been well characterized (Tiollais et al., Science 213:406-411, 1981; Tiollais et al., Nature 317:489-495, 1985; and Ganem and Varmus, Ann. Rev. Biochem. 56:651-693, 1987: see also EP0 278,940, EP0 241,021, WO 88/10301, and U.S. Pat. Nos. 4,696,898 and 5,024,938, which are hereby incorporated by reference) and encodes several distinct antigens, including three HB surface antigens (HBsAgs), an HB core antigen (HBcAg), an HBe antigen (HBeAg), and an HBx antigen (HBxAg) (see Blum et al., TIG 5(5):154-158, 1989). As will be evident to one of ordinary skill in the art, various immunogenic portions of the above-described HBV antigens may be combined on a single expression vector to induce an immune response. In addition, due to the large immunologic variability that is found in different geographic regions, particular combinations of antigens may be preferred for administration in particular geographic regions (Ouchterlony, Progr. Allergy 5:1, 1958: LeBouvier, J. Infect. 123:671, 1971; Bancroft et al., J. Immunol. 109:842, 1972; and Courouce et al., Bibl. Haematol. 42:1-158, 1976).

Within one embodiment of the invention, expression vectors encode, in addition to at least one disease associated antigen, a gene encoding an immunomodulatory cofactor, i.e., a factor which, when expressed in dendritic cells in addition to the disease associated antigen(s) causes the immune response to the antigen(s) to be enhanced in quality or potency from that which would have occurred in the absence of the cofactor. The quality or potency of a response may be measured by a variety of assays known to one of skill in the art including, for example, in vitro assays which measure cellular proliferation (e.g., ³H thymidine uptake), and in vitro cytotoxic assays (e.g., which measure ⁵¹Cr release) (see Warner et al., AIDS Res. and Human Retroviruses 7:645-655, 1991). In alternative embodiments, an immunomodulatory cofactor is, instead of be encoded by the expression vector, added exogenously either before, simultaneous with, or after the gene delivery vehicle is administered.

Immunomodulatory factors may be active both in vivo and ex vivo. Representative examples of such immunomodulatory factors include, for example, cytokines, such as IL-1, IL-2 (Karupiah et al., J. Immunology 144:290, 1990: Weber et al., J. Exp. Med. 166:1716, 1987; Gansbacher et al., J. Exp. Med. 172:1217, 1990: U.S. Pat. No. 4,738,927). IL-3, IL-4 (Tepper et al., Cell 57:503, 1989, Golumbek et al., Science 254:713, 1991 and U.S. Pat. No. 5,017,691), IL-5, IL-6 (Brakenhof et al., J. Immunol. 139:4116, 1987, and WO 90/06370), IL-7 (U.S. Pat. No. 4,965,195), IL-8, IL-9, IL-10, IL-11, IL-12 (Wolf et al., J. Immuno. 46:3074, 1991 and Gubler et al., PNAS 88:4143, 1991), IL-13 (WO 94/04680), IL-14, IL-15, α-interferon (Finter et al., Drugs 42(5):749, 1991, Nagata et al., Nature 284:316, 1980; Familletti et al., Methods in Enz. 78:387, 1981, Twu et al., PNAS USA 86:2046, 1989, Faktor et al., Oncogene 5:867, 1990, U.S. Pat. No. 4,892,743, U.S. Pat. No. 4,966,843, and WO 85/02862), β-interferon (Seif et al., J. Vir. 65:664, 1991), γ-interferons (Radford et al., The American Society of Hepatology 9:2008, 1991, Watanabe et al. PNAS 86:9456, 1989, Gansbacher et al., Cancer Research 50:7820, 1990, Maio et al., Can. Immunol. Immunother. 30:34, 1989, U.S. Pat. No. 4,762,791, and U.S. Pat. No. 4,727,138). G-CSF (U.S. Pat. Nos. 4,999,291 and 4,810,643), GM-CSF (WO 85/04188). tumor necrosis factors (TNFs) (Jayaraman et al., J. Immunology 144:942, 1990), CD3 (Krissanen et al., Immunogenetics 26:258, 1987), CD8, ICAM-1 (Altman et al., Nature 338:512, 1989; Simmons et al., Nature 331:624, 1988), ICAM-2 (Singer Science 255:1671, 1992), LFA-1 (Altmann et al., Nature 338:521, 1989), LFA-3 (Wallner et al., J. Exp. Med. 166(4):923, 1987), and other proteins such as HLA Class I molecules. HLA Class II molecules, B7 (Freeman et al., J. Immuno. 143:2714, 1989). B7-2, β₂-microglubulin (Parnes et al., PNAS 78:2253, 1981), chaperones, and MHC linked transporter proteins or analogs thereof (Powis et al., Nature 354:528, 1991). The choice of which immunomodulatory factor(s) to employ is based upon the therapeutic effects of the factor. Preferred immunomodulatory factors include α-interferon, γ-interferon, and IL-2.

Nucleic acid molecules encoding disease associated antigens can be readily obtained from a variety of sources, including for example, depositories such as the American Type Culture Collection (ATCC, Rockville, Md.), or from commercial sources such as British Bio-Technology Limited (Cowley, Oxford, England). Alternatively, cDNAs which encode such antigens may be generated from cells known to express or contain the corresponding gene (see U.S. Pat. Nos. 4,683,202; 4,683,195 and 4,800,159. See also PCR Technology: Principles and Applications for DNA Amplification, Erlich (ed.), Stockton Press, 1989). Alternatively, genes of known sequence, or which encode antigens of known amino acid sequence, may be synthesized, for example, on an Applied Biosystems Inc. DNA synthesizer (e.g., APB DNA synthesizer model 392 (Foster City, Calif.)).

C. Targeting Elements

As discussed above, one aspect of the present invention provides compositions and methods for targeting a gene delivery vehicle to a dendritic cell, either in vivo or in vitro. In the context of this invention, a targeting element is a molecule that has affinity for a molecule present on the surface of a dendritic cell. A wide variety of targeting elements can be utilized in the practice of this invention to specifically direct a gene delivery vehicle to a dendritic cell. As utilized herein, targeting elements are considered to be “capable of interacting with a molecule present on the surface” of a dendritic cell when a biological effect of the coupled targeting element may be seen in the cell, or, alternatively, when there is greater than at least about a 10-fold difference, and preferably greater than at least about a 25, 50, or 100-fold difference, between the binding of the targeting element to dendritic cells as compared to non-dendritic cells. Generally, it is preferable that the targeting element interact with a molecule present on the surface of the selected cell type with a K_{D} of less than about 10⁻⁵M, preferably less than about 10⁻⁶M, more preferably less than about 10⁻⁷M, and most preferably less than about 10⁻⁸M, as determined by Scatchard analysis (Scatchard, Ann. N.Y. Acad. Sci. 51:660, 1949). Suitable targeting elements are preferably non-immunogenic, not degraded by proteolysis, and not scavenged by the immune system. Particularly preferred targeting elements should have a half-life within an animal of between about 10 minutes and about 1 week.

Generally, targeting elements useful in the practice of this invention are proteins or peptides, although other non-proteinaceous molecules may also function as targeting elements. For example, antibodies (or the antigen binding domain thereof) can be utilized to target dendritic cells. Particularly useful antibodies are monoclonal antibodies which interact with the extracellular domains of integral membrane proteins found predominantly, or preferably exclusively, in the cell membranes of dendritic cells, (see generally, Wilchek and Bayer, Anal. Biochem 171:1-32, 1988). Standard techniques can be used to make antibodies to such proteins or other dendritic cell-specific markers. See generally Harlow and Lane. When the patient to be treated is human and the gene delivery vehicle is to be targeted in vivo, the antibody (or antigen binding thereof) employed is preferably humanized. Suitable markers for antibody generation include CD 11c, CD 54, CD 58, CD 25, CD 11a, CD 23, CD 32, CD 40, CD 1, CD 45, MHC Class I, MHC Class II, Mac-1, Mac-2, and Mac-3. These and other lineage specific markers can be used to separate or purify dendritic cells from more diverse cell populations. Both positive and negative selection strategies may be employed. Particularly preferred techniques for conducting cell selection include FACS and affinity chromatography.

Other suitable targeting elements include hormones, immune accessory molecules (e.g., B7, IL-2, α-interferon, and γ-interferon), cell adhesion molecules (e.g., ICAM-1, ICAM-2, and ICAM-3), and receptors known to be expressed on the surface of dendritic cells.

Alternatively, dendritic cell-specific ligands may be selected from libraries created utilizing recombinant techniques (Scott and Smith, Science 249:386, 1990; Devlin et al., Science 249:404, 1990; Houghten et al., Nature 354:84 1991; Matthews and Wells, Science 260:1113,1993; Nissim et al., EMBO J. 13(3):692-698, 1994), or equivalent techniques utilizing organic compound libraries.

In yet another alternative approach, gene delivery vehicles can be targeted to dendritic cells through the use of antibodies, preferably human antibodies when the patient to be treated is human, generated to the gene delivery vehicle or its components, including proteins, lipids, carbohydrates, nucleic acids, condensing agents, etc. The Fc portion of such an antibody will serve as the targeting element, targeting the gene delivery vehicle to dendritic cells that express Fc receptor molecules.

Once selected, suitable targeting elements may be produced by a variety of techniques. When the targeting element is a protein, it is preferably produced via recombinant techniques. Briefly, a gene, preferably a cDNA, encoding the targeting element is cloned into an appropriate expression vector, preferably one encoding a selectable marker. The particular vector selected will depend on the many factors, including, among others, the targeting element gene to be expressed and the host cell in which such expression will occur. The vector is then introduced by any of a variety of techniques into a suitable host cell. When possible, cells into which the vector was successfully introduced are selected. Cells are then cultured under appropriate nutrient conditions and the targeting element is expressed, preferably at high levels. The targeting element is then isolated and preferably purified from the cells or culture media, depending upon whether the protein is engineered to accumulate intracellularly or be secreted into the surrounding culture medium. As those in the art will appreciate, the particular vector, gene, host cells, culture conditions, and purification procedure(s) employed will vary depending upon the particular targeting element to be produced. As an alternative to recombinant protein production, particularly when the targeting element consists of a small polypeptide, solid state chemical synthesis techniques can be employed to produce sufficient quantities of the desired targeting element. Likewise, for non-proteinaceous targeting elements, chemical synthesis, isolation from natural sources, etc., may be employed to generate sufficient quantities of the desired targeting element.

1. High Affinity Binding Pairs

One embodiment of the invention concerns the use of a high affinity bind pair to target a gene delivery vehicle to a dendritic cell. Such methods typically comprise (a) administering to an animal a targeting element coupled to a first high affinity molecule of a high affinity binding pair, the coupled targeting element being capable of specifically binding to a dendritic cell, and (b) administering to the animal a gene delivery vehicle coupled to the second member of the high affinity binding pair, thereby targeting the gene delivery vehicle to a dendritic cell. As those in the art will appreciate, various dendritic cell targeting elements, affinity binding pairs, and gene delivery vehicles can be utilized. Representative examples of suitable affinity binding pairs include biotin/avidin (K_{D}=10⁻¹⁵M: Richards, Meth. Enz. 184:3-5, 1990, Green, Adv. in Protein Chem. 29:85, 1985); cytostatin/papain (K_{D}=10⁻¹⁴M; Bjork and Ylinenjarvi, Biochemistry 29:1770-1776, 1990): val-phosponate/carboxypeptidase A (K_{D}=10⁻¹⁴M; Kaplan and Bartlett, Biochemistry 30:8165-8170, 1991): 4CABP-RuBisCo (K_{D}=10⁻¹³M; Schloss, J. Biol. Chem. 263:4145-4150, 1988): and tobacco hornworm diuretic hormone/tobacco hornworn diuretic hormone receptor (K_{D}=10⁻¹¹M; Reagan et al., Arch. Insect Biochem. Physiol. 23:135-145, 1993). Either member (or molecule) of the high affinity binding pair may be coupled to the gene delivery vehicle (or conversely, to the targeting element). Nevertheless, the larger of the two members of the binding pair (e.g., avidin in the avidin/biotin pair) is preferably coupled to gene delivery vehicle.

Other high affinity binding pairs may also be developed, for example, by preparing and selecting antibodies for a selected antigen, and by further screening such antibodies to select those with a high affinity. Alternatively, antibodies or antibody fragments may also be produced and selected utilizing recombinant techniques. See generally co-owned U.S. Ser. Nos. 08/242,407 and 08/440,616.

2. Multifunctional Linking Agents

In other embodiments where targeting elements are employed, the targeting element is covalently linked to a gene delivery vehicle via a multifunctional linking agent. Multifunctional linking agents are molecules that contain at least two reactive groups separated by a spacer or “bridge.” In the practice of this invention, multifunctional linking agents are used to covalently bind a targeting element to a GDV. Upon activation of the reactive groups of the multifunctional linking agent in the presence of a GDV and a targeting element, covalent bonds are formed to link the GDV and targeting element together via the multifunctional linking agent. The spacer provides the spatial distance necessary to accommodate steric considerations of the moieties to be linked. Different linking agents may be selected based on the lengths of bridges desired for coupling.

In one method for selecting a desired multifunctional linking agent, a linking agent with a short spacer (4-8 Å) is used and the degree of linking between the GDV and the targeting element is determined. If linking is minimal or unsuccessful, a multifunctional linking agent with a longer spacer is then selected. This process may be repeated in an iterative pattern until a linking agent providing the minimum spacing necessary to avoid steric hindrance is identified.

A bifunctional linking agent that has identical reactive groups on either end of the bridge is said to be homobifunctional. Where the reactive groups are different, the bifunctional linking agent is referred to as a heterobifunctional. Examples of reactive groups include imidoesters, N-hydroxysuccinimidyl (NHS) esters, maleimides, pyridyl disulfides, carbodiimides, and arylazides, as well as others known in the art. The imidoesters and the NHS esters react with primary amines present on the GDV and targeting element, while maleimide and pyridyl disulfide react with sulfhydryl groups present on the GDV and targeting element. Carbodiimides couple carboxyl groups to primary amines present on the GDV and the targeting element. An arylazide is a photoactivatable group that forms reactive nitrene when exposed to ultraviolet or visible light at wavelengths ranging from 250-460 nm. The aryl nitrene thus formed reacts nonselectively to form a covalent bond.

In the present invention, the targeting element is covalently bound to the GDV utilizing a “multifunctional linking agent,” preferably a bifunctional linking agent (e.g., a homobifunctional or heterobifunctional linking agent). A variety of multifunctional linking agents may be utilized and are available through Pierce (Rockford, Ill.). Representative multifunctional linking agents include 4(4-N-maleimidophenyl)butyric acid hydride●HCl●½ dioxane (MPBH; a heterobifunctional non-cleavable linking agent), succinimidyl 4-(N-maleimidomethyl)cyclohexane-1-carboxylate (SMCC; a heterobifunctional linker), sulfosuccinimidyl (4(azidosalicylamido)hexanoate (sulfo-NHS-LC-ASA; a photoactivatable linking agent), sulfosuccinimidyl-2-[6-(biotinamido)-2-(p-azidobenzamido)-hexanoamido]ethyl-1,3′-dithiopropionate (Sulfo-SBED; a trifunctional linking agent having biotin covalently attached to a heterobifunctional reagent comprising a hydroxysuccinimido active ester and a photoreactive aryl azide), and disuccinimidyl suberate (DSS: a homobifunctional N-hydroxysuccinimdyl ester linker). Other multifunctional linking agents that may be utilized include, for example, N-succinimidyl-3-(2-pyridyl dithio) propionate (“SPDP”; Carlson et al., J. Biochem. 173:723, 1978), Sulfosuccinimidyl 4-N-maleimidomethyl) cyclohexane-1-carboxylate (“SulfoSMCC”), 1-ethyl-3 (3-dimethylaminopropyl) carbodiimide (“EDC”), and Bis-diazobenzidine (“BDB”). Methods for conjugation of a GDV to a targeting element via a multifunctional linking agent are provided in Example 2. below.

In another embodiment of the invention, the multifunctional linking agent further comprises a monosaccharide, disaccharide or an oligosaccharide wherein the carbohydrate is first covalently bound to a targeting element utilizing a linking agent described above. The modified targeting element is then covalently bound to a GDV via the carbohydrate moiety. In a preferred embodiment of this approach, a targeting element is bound to a aminated carbohydrate utilizing a multifunctional linking agent. For example, a homobifunctional linker such as DSS may be utilized to covalently bind the targeting element to the carbohydrate via amine groups present on the targeting element and the aminated carbohydrate. Alternatively, a heterobifunctional linker such as SMCC can be used to bind the carbohydrate to a sulfhydryl present on the targeting element to the amine group of the aminated carbohydrate. The modified targeting element may then be bound to the GDV. Briefly, the GDV and the modified targeting element are mixed at various pHs ranging from about 7.4 to about 8.4 and incubated, preferably overnight at about 4° C. Following incubation, the mixture is treated with sodium cyanoborohydride. The reaction mixture is dialyzed at low temperature (about 2° C. to 10° C.) for a sufficient time (about 1 to 48 hours) to remove cyanoborohydride and sterilized by passage through an appropriate filter. Alternative procedures may be employed, depending on the carbohydrate and linker employed, as those in the art will appreciate.

As will be appreciated by those skilled in the art, other targeting mechanisms which enable dendritic cell targeting also fall within the scope of the invention.

D. GDV Production

Once the GDV has been designed, it must be produced in an amount sufficient for conjugation to a desired targeting element and/or for administration to an animal. If the GDV is a recombinant viral vector, it may be produced utilizing a packaging system. A variety of viral vector packaging systems are described below in which one or more essential functions of the parent virus has been deleted so that it is deficient in some function (e.g., genome replication), but retains a packaging signal and the ability to express gene products from one or more nucleic acid molecules. Representative examples of viral vector packaging systems include those for retroviral vectors, alphaviral vectors and adenoviral vectors. The deleted essential function or functions are provided by packaging cells into which the vector genome can be introduced to yield producer cell lines that then make viral particles encapsidating the recombinant viral vector. In preferred embodiments, such producer cell lines produce viral vectors substantially free from contamination with replication competent virus. The vector genome is then introduced into target cells by an infection event (“transduction”) but is incapable of further propagation. In any such situation, it is important to prevent the recombination of the various parts of the virus in a producer cell line to give replication competent virus genomes, or to eliminate cells in which this occurs. The expression vector may be readily assembled from any virus utilizing standard recombinant techniques (e.g., Sambrook et. al., Molecular Cloning: A Laboratory Manual, 2d ed. Cold Spring Harbor Laboratory Press, 1989). Further description of the construction of retroviral vectors is described in U.S. Ser. No. 07/586,603, herein incorporated by reference.

Within one embodiment of the present invention, the GDVs are retroviral vectors. Typically, such vectors comprise a 5′ LTR, a tRNA binding site, a packaging signal, one or more genes of interest, an origin of second strand DNA synthesis, and a 3′ LTR, wherein the vector lacks gag/pol or env coding sequences. As utilized herein, a 5′ LTR should be understood to include a 5′ promoter element and sufficient LTR sequence to allow reverse transcription and integration of the DNA form of the vector. The 3′ LTR includes a polyadenylation signal and sufficient LTR sequence to allow reverse transcription and integration of the DNA form of the vector (see U.S. Ser. No. 07/395,932 and U.S. Ser. No. 07/565,606).

Preferably, recombinant retroviral vectors are provided which lack both gag/pol and env coding sequences. As utilized herein, the phrase “lacks gag/pol or env coding sequences” should be understood to mean that the recombinant retroviral vector does not contain at least about 20, preferably at least about 15, more preferably at least about 10, and most preferably, at least about 8 consecutive nucleotides which are found in gag/pol or env genes used to construct packaging cell lines. Representative recombinant retroviral vectors are set forth in more detail below and in Example 1.

As an illustration, construction of recombinant retroviral vectors which lack gag/pol and/or env coding sequences may be accomplished by preparing vectors which lack an extended packaging signal, i.e., a nucleotide sequence beyond the minimum core sequence required for packaging that allows increased viral titer due to enhanced packaging. For example, the minimum core packaging signal for the Moloney murine leukemia virus (MoMLV) begins approximately at nucleotide 212 and continues through the Pst I site at nucleotide 567 (Shinnick et al., Nature 293:543, 1981 and Mann., et al., Cell 33:153, 1983). The extended packaging signal of MoMLV extends through the start of the gag/pol gene at nucleotide 621 and beyond nucleotide 1.560. Thus, recombinant MoMLV retroviral vector constructs which lack an extended packaging signal may be constructed by truncating the packaging signal after nucleotide 567 but before the gag initiation codon, with truncations terminating after nucleotide 570, 575, 580, 585, 590, 595, 600, 610, 615 or 617 being preferred.

When such recombinant retroviral vectors are utilized, it is preferable to utilize packaging cell lines for producing viral particles wherein at least the codons of 5′ terminal end of the gag/pol gene are modified to take advantage of the degenerate nature of the genetic code to minimize the possibility of homologous recombination between the vector and sequences in the packaging cell coding for the viral structural proteins. Additional techniques for reducing the possibility of recombination events between vectors present in a packaging cell and the recombinant retroviral genome to be packaged are provided in the co-owned U.S. Ser. No. 08/240,030 and U.S. Ser. No. 08/305,699.

Packaging cell lines suitable for use with the above described recombinant retroviral vectors may be readily prepared using techniques known in the art (see U.S. Ser. No. 08/240,030, filed May 9, 1994; see also U.S. Ser. No. 07/800,921, filed Nov. 27, 1991), and utilized to create producer cell lines for the production of recombinant vector particles.

In a further embodiment of the invention, alphavirus packaging cell lines are provided. In particular, alphavirus packaging cell lines are provided wherein the viral structural proteins, supplied in traits from one or more stably integrated expression vectors, are able to encapsidate transfected, transduced, or intracellularly produced vector RNA transcripts in the cytoplasm and release infectious, packaged vector particles through the cell membrane, thus creating an alphavirus vector producing cell line. Alphavirus RNA vector molecules, capable of replicating in the cytoplasm of the packaging cell, can be produced initially utilizing, for example, an SP6 or T7 RNA polymerase system to transcribe in vitro a cDNA vector clone encoding the recombinant alphaviral genome which comprises the gene(s) of interest and the alphavirus non-structural proteins. Vector RNA transcripts are then transfected into the alphavirus packaging cell line such that the vector RNA replicates to high levels and is subsequently packaged by viral structural proteins, yielding infectious vector particles.

In an effort to optimize vector producing cell line performance and titer, two successive cycles of gene transfer may be performed. In particular, rather than directly transfecting alphavirus RNA vector molecules into the final producing cell line, the vector may first be transfected into a primary alphavirus packaging cell line. The transfected primary packaging cell line releases infectious vector particles into the culture supernatants and these vector-containing supernatants are subsequently used to transduce a fresh monolayer of alphavirus packaging cells. Transduction into the final alphavirus vector producing cells is preferred over transfection because of its higher RNA transfer efficiency into cells and optimized biological placement of the vector in the cell. This leads to higher expression and higher titer of packaged infectious recombinant alphavirus vector.

Packaging cell lines suitable for use with the above described alphaviral vector constructs may be readily prepared (see U.S. Ser. No. 08/404,796, filed Mar. 15, 1995; see also U.S. Ser. No. 08/376,184 filed Jan. 18, 1995, U.S. Ser. No. 08/405,627 filed Mar. 15, 1995, and WO 95/07994).

Within further embodiments of the invention, adenovirus packaging cell lines are provided. Adenovirus vectors are derived from nuclear replicating viruses and may be constructed such that they are replication defective. One or more nucleic acid molecules may be carried by adenoviral vectors for delivery to target cells (see Ballay et al., EMBO J. 4:3861, 1985, Thummel et al., J. Mol. Appl. Genetics 1:435, 1982 and WO 92/05266).

Within another embodiment of the invention, a targeted gene delivery vehicle may include one or more fusigenic proteins to assist in gene delivery. Representative fusigenic proteins include ecotropic murine retrovirus envelope proteins, other retrovirus envelope proteins modified to disable normal receptor recognition, fusigenic proteins from herpes simplex virus fusigenic proteins gH and gL, Epstein-Barr virus fusigenic proteins, measles virus proteins, malarial sporozoite fusigenic proteins, and other proteins known in the art to have fusogenic properties.

E. Purification of Gene Delivers Vehicles

Once the GDVs are produced, they are preferably purified prior to conjugation to the desired targeting element. In addition, compositions comprising targeted GDVs are preferably purified again prior to administration. The techniques utilized for purification is dependent on the type of GDV to be purified. For example, there are a variety of techniques known in the art which may be used if the GDV is an enveloped recombinant viral vector, a nucleic acid or a liposome. A preferred method is described below and is particularly useful in the isolation and purification of recombinant retroviral and alphaviral particles, as well as being applicable to the isolation and purification of other enveloped viruses. A sulfated oligosaccharide solid phase column matrix may be utilized in a variety of formats to purify enveloped viral vectors. Briefly, a sulfated oligosaccharide is placed in a column, a preparation containing an enveloped virus is passed through the column, and the virus adsorbs to the sulfated oligosaccharide. It will be appreciated by those in the art that optimization of column performance is dependent upon variables such as virus type, sample volumes, and flow rates, among others. Substances in the preparation that do not bind to the sulfated oligosaccharide (e.g., albumin, pyrogens and DNA), will pass through the column. The virus is then de-absorbed from the sulfated oligosaccharide by passing a high ionic strength buffer (i.e., about 0.5M-3.0M salt in a buffered solution) over the matrix. Prior to elution of the virus, it may be desirable to wash the column one or more times with another buffer solution (e.g., one having an ionic strength greater than the application buffer strength, but less than that required to elute the virus) to remove any residual substances that are unbound or weakly bound to the column. As will be appreciated, purification of an enveloped virus utilizing a sulfated oligosaccharide may be performed in combination with other purification techniques, such as standard chromatographic procedures, which include, for example, ion exchange chromatography and gel filtration chromatography. In a multi-step procedure, a step utilizing a sulfated oligosaccharide may be the initial step, final step, or be interposed between other steps, and may be repeated (see U.S. Ser. No. 08/093,436).

As an alternative to column purification, a batch format may be utilized. Briefly, a sulfated oligosaccharide can be added directly to a virus-containing preparation. To maximize the sulfated oligosaccharide/virus interaction, the mixture is gently agitated. Following virus adsorption (e.g., 30 minutes at room temperature with gentle stirring), the unbound or weakly bound portion of the preparation can be separated from the virus containing sulfated oligosaccharide in a variety of ways. For example, the sulfated oligosaccharide may be separated by centrifugation (e.g., 1000×g for 5 minutes), filtration or settling (e.g., 2 hour at 1×g). Virus bound to the sulfated oligosaccharide is de-adsorbed using a high ionic strength buffer. Preferably, the sulfated oligosaccharide is washed with one or more solutions prior to elution of the virus (see U.S. Ser. No. 08/093,436). The GDVs are typically purified to a level ranging from 0.25% to 25%, and preferably about 5% to 20% before conjugation.

In addition, if the GDV is a nucleic acid, there are a variety of techniques known in the art including, for example, purification by CsCl-ethidium bromide gradient, ion-exchange chromatography, gel-filtration chromatography, and differential precipitation with polyethylene glycol. Further description of the purification of nucleic acids is provided in Sambrook et. al., Molecular Cloning: A Laboratory Manual, 2d ed. (Cold Spring Harbor Laboratory Press, 1989).

When the GDV is a liposome, a variety of purification methods known to those skilled in the art may be utilized and are described in more detail in Mannino and Gould-Fogerite (BioTechniques 6:682, 1988). Briefly, preparation of liposomes typically involves admixing solutions of one or more purified phospholipids and cholesterol in organic solvents and evaporating the solvents to dryness. An aqueous buffer containing the GDVs is then added to the lipid film and the mixture is sonicated to create a fairly uniform dispersion of liposomes. In certain embodiments, dialysis, gel filtration, or ultracentrifugation is then be used to separate unincorporated components from the intact liposomes. (Stryer, L., Biochemistry, pp:236 1975 (W. H. Freeman, San Francisco): Szoka et al., Biochim. Biophys. Acta 600:1, 1980; Bayer et al., Biochim. Biophys. Acta. 550:464, 1979; Rivnay et al., Meth. Enzymol. 149: 119, 1987; Wang et al., PNAS 84: 7851, 1987 and, Plant et al., Anal. Biochem. 176:420, 1989.

F. GDV/Targeting Element Production

Several linking agents may be utilized to bind target elements to GDV. The methods used vary depending on the available functional groups on the exterior of the GDV and the targeting element. For example, if the both the targeting element and the GDV have primary amines available on their surface a multifunctional linking agent such as disuccinimidyl suberate (DSS, Pierce, Rockford, Ill.) which is a homobifunctional N-hydroxysuccinimdyl ester linking agent. If however, the GDV contains sulfhydryl functional groups on its surface and the targeting element has a primary amine available a heterobifunctional linking agent may be utilized for example succinimidyl 4-(N-maleimidomethyl) cyclohexane-1-carboxylate. If the GDV contains a carboxyl functional group and the targeting element has a sulfhydryl functional group then a heterobifunctional group such as 4(4-N-maleimidophenyl)butyric acid hydride●HCl●½ dioxane may be utilized. Other linking agents may be utilized include trifunctional linking agents which permit binding of a targeting element, a GDV and a third element and light activated linking agents see Example 4 below. Those in the art will appreciate GDV can be bound to targeting elements by a variety of other methods.

G. Purification of GDV/Targeting Element

Several methods may be utilized for the purification of the GDV/targeting element including, for example, molecular sieve column chromatography (e.g., Sephadex® or Sephacryl®), affinity chromatography, HPLC, R-HPLC. equilibrium centrifugation (i.e., cesium chloride gradient centrifugation), and sucrose density gradients. Procedures for conducting such purifications are known in the art, and will be appreciated by those in the art, will likely vary depending upon the particular product being purified.

H. Formulation

Following purification of a composition comprising a targeted GDV, the preparation is preferably formulated into a pharmaceutical composition. Such compositions may be in liquid or dry form. Preferred liquid compositions comprise aqueous solutions in which the desired product is suspended in a pharmaceutically acceptable carriers and/or diluent and may additionally comprise stabilizers, and other excipients. Alternatively, the compositions of the invention can be prepared in solid formulations intended for resuspension just prior to use. Dry formulations include those which are lyophilized or freeze dried.

When GDV is a lipid enveloped virus, preferred dry formulations comprise some or all of the following: one or more pharmaceutically acceptable carriers and/or diluents; a saccharide; a high molecular weight structural additive; a buffering component; water; and one or more amino acids. The combination of some or all of these components acts to preserve the activity of the targeted GDV upon freezing and lyophilization, or drying through evaporation. Pharmaceutically acceptable carriers or diluents according to the invention are non-toxic to recipients at the dosages and concentrations employed. Representative examples of carriers or diluents for injectable solutions include for example water, isotonic saline solutions (i.e., phosphate-buffered saline or Tris-buffered saline, preferably buffered at physiological pH), mannitol, dextrose, glycerol, and ethanol, as well as polypeptides or proteins such as human serum albumin.

The saccharide provides, among other things, support in the lyophilized or dried state. Although the preferred saccharide is lactose, other saccharides may be used, such as sucrose, mannitol, glucose, trehalose, inositol, fructose, maltose or galactose. In addition, combinations of saccharides can be used, for example, lactose and mannitol, or sucrose and mannitol. A particularly preferred concentration of lactose is 3% to 4% by weight. Preferably, the concentration of the saccharide ranges from 1% to 12% by weight.

The high molecular weight structural additive aids in preventing viral aggregation during freezing and provides structural support in the lyophilized or dried state. Within the context of the present invention, structural additives are considered to be of “high molecular weight” if they are greater than 5000 molecular weight. A preferred high molecular weight structural additive is human serum albumin. However, other substances may also be used, such as hydroxyethyl-cellulose, hydroxymethyl-cellulose, dextran, cellulose, gelatin, or povidone. A particularly preferred concentration of human serum albumin is 0.1% by weight. Preferably, the concentration of the high molecular weight structural additive ranges from 0.1% to 10% by weight.

The amino acid(s), if present, function to further preserve viral infectivity upon cooling and thawing of the aqueous suspension. In addition, amino acids function to further preserve viral infectivity during sublimation of the cooled aqueous suspension and while in the lyophilized state. A preferred amino acid is arginine, but other amino acids such as lysine, ornithine, serine, glycine, glutamine, asparagine, glutamic acid or aspartic acid can also be used. A particularly preferred arginine concentration is 0.1% by weight. Preferably, the amino acid concentration ranges from 0.1% to 10% by weight.

The buffering component acts to buffer the solution by maintaining a relatively constant pH. A variety of buffers may be used, depending on the pH range desired, preferably between 7.0 and 7.8. Suitable buffers include phosphate buffer and citrate buffer. A particularly preferred pH of the recombinant virus formulation is 7.4, and a preferred buffer is tromethamine.

In addition, it is preferable that the aqueous solution contain a neutral salt which is used to adjust the final formulated recombinant retrovirus to an appropriate iso-osmotic salt concentration. Suitable neutral salts include sodium chloride, potassium chloride or magnesium chloride. A preferred salt is sodium chloride.

A preferred lyophilized composition for lipid enveloped viruses, particularly recombinant retroviruses and alphaviruses, comprises 10 mg/mL mannitol, 1 mg/mL HSA, 20 mM Tris, pH 7.2, and 150 mM NaCl being particularly preferred (see U.S. Ser. No. 08/153,342). Such compositions are stable at −70° C. for at least six months. The pharmaceutical compositions of the invention may also additionally include factors to stimulate cell division, and hence, uptake and incorporation of the administered GDVs. Particularly preferred methods and compositions for preserving recombinant viruses are described in U.S. Ser. No. 08/135,938, U.S. Ser. No. 08/122,791, and U.S. Ser. No. 08/367,071). In liquid or solid form, after preparation of the composition where the GDV is a recombinant virus, the recombinant virus preferably will constitute about 10 ng to 1 mg of material per dose, with about 10 times this amount of material present as copurified contaminants. Preferably, the composition is administered in doses of about 0.1 to 1.0 mL of aqueous solution, which may or may not contain one or more additional pharmaceutically acceptable excipients, stabilizers, or diluents.

1. Administration

Compositions according to the invention, be they dendritic cells modified ex vivo or GVDs for direct administration, are typically administered in vivo via parenteral (e.g., intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue, such as the liver, bone marrow, or into the tumor in the case of cancer therapy. Non-parenteral routes are discussed further in U.S. Ser. No. 08/366,788, filed Dec. 3, 1994.

Preferably, the composition is administered to an animal via the desired route and then the animal is tested for the desired biological response. Such testing may include immunological screening assays e.g., CTL assays, antibody assays. The test(s) performed will depend on the product produced by the nucleic acid molecule introduced by the targeted GDV and the disease to be treated or prevented. On the basis of the results of such testing, the titers of the targeted GDVs to be administered may be adjusted to further enhance the desired effect(s) if more than administration is required.

Administration by many of the routes of administration described herein or otherwise known in the art may be accomplished simply by direct administration using a needle, catheter or related device, at a single time point or at multiple time points. In addition, an “administration” of a gene delivery vehicle (or ex vivo transduced cells, for that matter) at a given time point includes administration to one or more areas, or by one or more routes. In certain embodiments of the invention, one or more dosages is administered directly in the indicated manner: intravenously at dosages greater than or equal to 10³, 10⁴, 10⁵, 10⁶, 10⁷, 10⁸, 10⁹, 10¹⁰ or 10¹¹ cfu; intraarterially at dosages greater than or equal to 10³, 10⁴, 10⁵, 10⁶, 10⁷, 10⁸, 10⁹, 10¹⁰ or 10¹¹ cfu; intramuscularly at dosages greater than or equal to 10³, 10⁴, 10⁵, 10⁶, 10⁷, 10⁸, 10⁹, 10¹⁰ or 10¹¹ cfu. with dosages of 10¹⁰ or 10¹¹ cfu being preferred: intradermally at dosages greater than or equal to 10³, 10⁴, 10⁵, 10⁶, 10⁷, 10⁸, 10⁹, 10¹⁰ or 10¹¹ cfu; pulmonarily at dosages greater than or equal to 10³, 10⁴, 10⁵, 10⁶, 10⁷, 10⁸, 10⁹, 10¹⁰ or 10¹¹ cfu; subcutaneously at dosages greater than or equal to 10³, 10⁴, 10⁵, 10⁶, 10⁷, 10⁸, 10⁹, 10¹⁰ or 10¹¹ cfu, with dosages of 10⁹, 10¹⁰ or 10¹¹ cfu being preferred; interstitially at dosages greater than or equal to 10³, 10⁴, 10⁵, 10⁶, 10⁷, 10⁸, 10⁹, 10¹⁰ or 10¹¹ cfu, with dosages of 10⁸, 10⁹, 10¹⁰ or 10¹¹ cfu being preferred; into a lymphoid organ such as the spleen, a tonsil, or a lymph node at dosages greater than or equal to 10³, 10⁴, 10⁵, 10⁶, 10⁷, 10⁸, 10⁹, 10¹⁰ or 10¹¹ cfu; into a tumor at dosages greater than or equal to 10³, 10⁴, 10⁵, 10⁶, 10⁷, 10⁸, 10⁹, 10¹⁰ or 10¹¹ cfu, with dosages of 10⁸, 10⁹, 10¹⁰ or 10¹¹ cfu being preferred; intraperitoneally at dosages greater than or equal to 10³, 10⁴, 10⁵, 10⁶, 10⁷, 10⁸, 10⁹, 10¹⁰ or 10¹¹ cfu, with dosages of 10⁸, 10⁹, 10¹⁰ or 10¹¹ cfu being preferred; and into the afferent lymph at dosages greater than or equal to 10³, 10⁴, 10⁵, 10⁶, 10⁷, 10⁸, 10⁹, 10¹⁰ or 10¹¹ cfu. For purposes of the convenience, “cfu” shall also refer to non-viral particles, such that one cfu is equivalent to one non-viral particle.

Other routes and methods for administration include non-parenteral routes, such as are disclosed in co-owned U.S. Ser. No. 08/366,788, as well as administration via multiple sites, as disclosed in co-ow ned U.S. Ser. No. 08/366,784.

### EXAMPLES

The following examples are included to more fully illustrate the present invention. Additionally, these examples provide preferred embodiments of the invention and are not meant to limit the scope thereof. Standard methods for many of the procedures described in the following examples, or suitable alternative procedures, are provided in widely reorganized manuals of molecular biology, such as, for example “Molecular Cloning.” Second Edition (Sambrook, et al., Cold Spring Harbor Laboratory Press, 1987) and “Current Protocols in Molecular Biology” (Ausubel, et al., eds. Greene Associates/Wiley Interscience, NY. 1990).

### Example 1

### Identification of Dendritic Cells as Key Antigen Presenting Cells Following Direct Administration of Gene Delivery Vehicles

As discussed above, direct injection of recombinant retroviruses carrying an expression vector coding for at least one disease associated antigen vector is preferred to ex-vivo approaches. However, compared to an ex vivo approach where the desired disease associated antigen is expressed by transduced cells., e.g., fibroblasts, which are then administered to a patient, direct administration of gene delivery vehicles to a patient lead to transduction of cells at and adjacent to the injection site, as well as transducing cells residing in tissues where the gene delivery vehicle can be transported, such as to lymphoid tissue following intravenous or subcutaneous administration. Previously, it was unknown which cells were responsible for presenting the disease associated antigen(s) encoded by the expression vector directly delivered by injection of gene delivery vehicles. Identification of transduced cells which present antigen(s) encoded by an expression vector delivered by direct injection to an animal is described below.

In previous biolocalization studies designed to trace expression vectors delivered by a recombinant retrovirus directly injected into animals, whole organ PCR was used to detect proviral DNA at the site of injection (Sajjadi, et al., 1994; Kamantigue, et al., 1995) In addition, the proviral DNA was sometimes detected at secondary lymphoid organs, a result consistent with the observation that these animals mounted excellent immune responses to the immunogen encoded by the provirus.

In this study, four expression vectors delivered using recombinant retroviruses, each encoding either HIV gp160/120 envelope, bacterial β-galactosidase, chicken ovalbumin, or luciferase, were used to identify cells involved in antigen presentation and immune induction following direct administration. Four cell populations were examined. The first cell population contains proviral copies of the expression vector (Group I), as determined PCR. The second subset of cells are those which express the encoded protein (Group II). β-gal histochemistry and immunohistochemistry assays were employed to analyze these cells. The third subset (Group III) consists of subpopulations of Group II cells that can present antigenic peptides to the immune system. Group III cells were identified by their ability to induce immune responses in T cell assays. Finally, Group IV consists of a distinct group of cells which do not contain proviral copies of the expression vector and thus do not express the antigenic protein or peptide from its corresponding gene, but nonetheless present the antigen(s). Examples of Group IV cells include phagocytic cells which engulf primary transduced cells or their fragments. Such cells then reprocess and present antigenic peptides in the context of their own MHC molecules.

Retroviral Vectors

Non-replicating, amphotropic murine recombinant retroviruses carrying expression vectors encoding HIV gp 160/120, bacterial β-galactosidase, luciferase and chicken ovalbumin were used. Retroviral vector backbones and high titer viral preparations (>1×10⁷ cfu/mL) were generated as described in WO 89/09271, WO 92/05266, and co-owned U.S. Ser. No. 08/367,071. A 1.2 kb Bam HI to Bgl II fragment encoding a cDNA of chicken ovalbumin in pUG-1 vector (Moore, et al., 1993) was cloned into the Bam HI site of pSP73 (Promega, Madison Wis.). A 1.2 kb Xho I to Cla I fragment was recovered and substituted for the corresponding 3.8 kb Xho I to Cla I fragment from N2-HIV gp160/120. Aliquots of the viral preparations were tested for replication-competent retrovirus (RCR) and determined to be RCR-free.

Animals and Vector Immunizations

Six- to eight- weeks old female BALB/c (H-2^{d}) or C57BL/6 (H-2^{b}) mice from Harlan Sprague-Dawley were used in all experiments. On days 1, 4 and 7, mice were injected either intramuscularly (i.m.) in both gastrocnemius or Tibialis anterior muscles with 100 μl/injection, for a total of 200 μl/mouse or intradermally at the base of tail with 100 μl of the retroviral preparations. Spleens were harvested and in vitro CTL cultures were initiated by mixing with appropriate irradiated splenocytes.

Immunohistochemistry

Antibodies to the following lymphocyte markers were used as culture supernatants or purified immunoglubulins: CD4 (rat IgG): CD8 (rat IgM): Macrophages (rat IgG): and B220 (rat (IgM). Commercially available biotinylated antibodies to CD45 (30R11.1. rat IgG: Pharmingen, San Diego, Calif.). rat Ig (polyclonal; Jackson ImmunoResearch Labs., Inc., West Grove, Pa.), and rabbit Ig (polyclonal: Jackson ImmunoResearch, PA) were also used. Normal rabbit serum was used as a negative control.

Muscles from injection sites were carefully excised, immersed in OCT and rapidly frozen in liquid nitrogen and stored at −70° C. until use. Cryostat sections at 5-6 μm were cut, air dried and fixed in acetone for 5 min. Sections were incubated with previously titrated optimal concentrations of antibodies diluted in Tris-buffered saline (pH 7.4) for 60 min., washed in Tris-buffered saline, incubated with biotinylated anti-rat or rabbit IgG, and washed and then incubated with streptavidin conjugated alkaline phosphatase (Jackson ImmunoResearch. PA). Bound alkaline phosphatase was detected with the substrate Fast-violet, as previously described (Surh, J. Exp. Med., vol. 176:495-505). The sections were lightly counterstained with Meyer's hematoxylin and photographed under a Zeiss Axioscope microscope. To minimize background, antibodies were used after spinning in a Airfuge ultracentrifuge (Beckman Instruments, Inc., Palo Alto, Calif.) at 148,000×g for 30 min, and discarding the pellet.

Isolation of Infiltrating Cells

Injection sites were visualized via fluorescent emission from co-injected latex beads followed by excision and dicing with surgical scissors. Resulting small pieces of muscle (average sizes of 0.5 cm³) were digested with trypsin at 37° C. for 1 hr. with occasional shaking. At the end of the digestions, fetal calf serum was added and residual muscle pieces were separated by gravity. Infiltrating cells were washed with media and counted followed by luciferase and B3.Z assays.

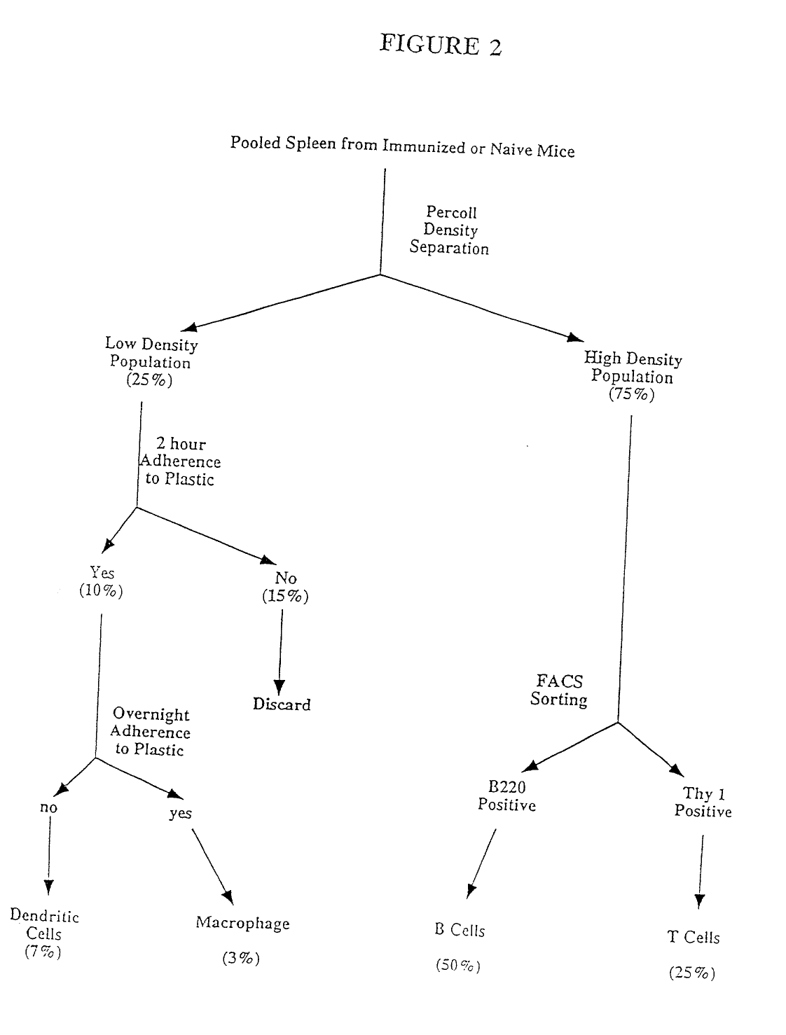

Splenocyte Fractionation by Percoll Density Gradients (see FIG. 2)

Percoll stock was prepared by mixing 9 parts Percoll to 1 part 10× phosphate buffered saline (PBS). “1.030” and “1.075” working Percoll solutions were made by mixing 75% PBS plus 25% Percoll stock solutions and 40% PBS plus 60% Percoll stock solutions, respectively. Splenocytes from naive or immunized animals were fractionated into B plus T cells, macrophages and dendritic cells by following methods. Spleens were mashed in DNAse (Calbiochem. 3800 units/mL) in Hank's balanced salt solutions (HBSS) at room temperature with occasional rocking for 1 hour, followed by filtering through nylon mesh to remove debris. Resulting cells were pooled and centrifuged at 1500 rpm for 5 min., followed by resuspension in “1.075” Percoll solutions. “1.075” Percoll solutions containing the pooled splenocytes were carefully overlayed with “1.030” Percoll solutions and the gradients were spun for 20 min. at 1200 rpm. After the Percoll centrifugation, low density cells in the interface and high density cells in the pellet were separately recovered and washed extensively. High density cells included red blood cells, T and most of the B cells except plasma cells. Low density cells included some B cells (including plasma cells), dendritic cells, monocytes and macrophages, but no T cells as judged by FACS. Most of the B cells in low density fractions were removed by careful washing after incubating cells for 2 hr. in tissue culture plates with complete medium (RPM11640, 10% fetal bovine serum). Remaining adherent cells, which included dendritic cells, monocytes and macrophages, were further incubated overnight. Mature macrophages were separated from all other cell types since they are the only cells which still adhere after overnight culture.

X-gal (5-bromo-4-chloro-3-indosyl b-D-galactopyranoside) Staining

Cultures were fixed with 3% formaldehyde for 5 min. at room temperature, followed by washing with PBS. The cells were overlayed with a solution of 1 mg/mL X-gal/mL, 5 mM potassium ferrocyanide, 5 mM of potassium ferricyanide, and 2 mM MgCl₂. The plates were examined microscopically for the presence of LacZ expressing, blue cells after an overnight incubation at 37° C.

In vitro CTL Induction and Cytotoxicity Assays

Five mice were immunized by administering recombinant retroviruses carrying expression vectors encoding HIV gp160/120. B+T cells, dendritic cells and macrophage fractions were prepared. Five non-immunized litter mates were processed in an analogous manner. Cell fractions were mixed with irradiated stimulators at a 50:1 ratio and cultured for 7 days. ⁵¹Cr release assays (Warner, et al. (1991), AIDS Res. and Human Retroviruses, vol. 7:645-655) were performed and percent lysis was calculated.

Ex vivo Transduction of Dendritic Cells and Monocytes and Adoptive Transfer

Naive mice were sacrificed and low density fractions were prepared as described above and put into in media containing GM-CSF (25 units/mL) and IL-4 (100 units/mL). DA/KT-1 and DA/β-gal producer cell lines were seeded in transwells with pore sizes of 0.45 micron. Dendritic cells and producer cells in transwells were co-cultured for three days. Recovered cells were extensively washed. Naive recipient mice were injected intraperitoneally with these transduced dendritic cells or fibroblasts stably transduced with HIV gp160/120. Spleens from recipient mice were harvested 7 days later, followed by in vitro restimulation and ⁵¹Cr release assays.

B3.Z Assays

B3.Z cells are T cell hybridomas which recognize chicken ovalbumin in the context of H-2K^{b} molecules. They contain a plasmid containing the bacterial LacZ gene fused to the minimal promoter of the human IL-2 gene, and are therefore LacZ inducible upon antigenic stimulation. Antigen presenting cells are either cells transfected with ovalbumin gene or fractionated splenocytes from ovalbumin expression vector immunized mice. Individual cultures containing 2×10⁵ B3.Z cells and 1×10⁷ antigen presenting cells were incubated overnight, followed by X-gal staining to assess the level of B3.Z activation. Numbers of antigen presenting cells in each fraction were estimated from the standard curve generated from cultures containing known numbers of cells transfected with ovalbumin genes.

Results

To define the cell types which are transduced and induce immunity following direct administration to muscle of recombinant retrovirus-based gene delivery vehicles, injection sites were examined by immunohistochemisty to identify in vivo transduced cells. Mice were immunized with a recombinant retrovirus carrying an expression vector encoding chicken ovalbumin. After three injections, mice were sacrificed for cryosection and the injection sites were analyzed by immunohistochemistry with antibodies against a variety of leukocyte markers and ovalbumin.

Many infiltrating cells were found in the endomesium near the injection sites. From the marker profile analyses, F4/80 positive monocytes/macrophages were identified as the most prominent infiltrating cells at the injection sites. Additionally, a significant number of CD4 positive cells and occasional B cells infiltrated at the injection sites. No CD8 positive cells were found. Two dendritic cell-specific antibodies against distinct epitopes. NLDC-145 and N418, enabled detection of significant numbers of dendritic cells at the injection sites. Mice injected with formulation buffer only showed comparable lymphocyte infiltration, suggesting the needle injury from injection is sufficient enough to cause lymphocyte infiltration. When the intradermal route of injection (which induces comparable immune responses) was examined, injection sites showed comparable infiltration by cells with the same phenotypes.

By immunohistochemistry some infiltrating cells were found to express ovalbumin proteins. Double staining with anti-ovalbumin and F4/80 antibodies indicated that some, but not all, ovalbumin positive cells were co-stained with the macrophage marker, F4/80. To verify injection site expression of proteins encoded by the expression vectors, infiltrating cells were isolated from animals injected with luciferase- or ovalbumin-encoding gene delivery vehicles. Cells were first analyzed by NBT assays, verifying that most of them are indeed of the macrophage/monocyte lineage. Next, infiltrating cells were subjected to either luciferase or B3.Z assays. Luciferase assays revealed that most of the luciferase activity was concentrated in infiltrating cells, but not in residual muscles. Furthermore, B3.Z assays indicate that the infiltrating cells present ovalbumin-derived peptides to T cells. No staining was found using anti-ovalbumin antibodies in parallel sections with normal rabbit serum or at the injection sites in mice treated with HIV gp160/120-encoding gene delivery vehicles. These results indicate that some fraction of infiltrating cells at the injection sites were indeed transduced and expres antigenic proteins which can be processed to stimulate immune responses.

From these experiments, inflammation caused by needle injury likely promotes the infiltration of leukocytes which then become transduced by gene delivery vehicles. These transduced leukocytes then migrate to secondary lymphoid organs to elicit immune responses. To confirm this, proviral DNA integration and protein expression was analyzed in various leukocyte populations in lymph nodes and spleens.

Previous biolocalization studies using DNA isolated from whole organs led to detection of proviral DNA in some secondary lymphoid organs (Sajjadi, et al., supra; Kamantigue et al., supra). Because of the limited sensitivity of whole organ PCR, PCR assays were performed on fractionated spleen cells. Splenocytes from immunized mice were pooled and separated into B cells, T cells, macrophages and dendritic cell fractions. In mice immunized with genes coding for HIV gp160/120 or β-gal, in addition to a marker gene conferring resistance to neomycin, the neo markers encoded by both vectors were detected in the dendritic and macrophage cell fractions.

Since proviral DNA integration was detected in leukocyte cell fractions, expression vector-derived protein expression was also analyzed in these transduced cell fractions. BALB/c mice were injected three times with gene delivery vehicles directing β-gal expression. Their spleens were pooled and fractionated as described above, followed by X-gal staining. In agreement with PCR data, unfractionated spleen and dendritic cell fractions showed β-gal staining. Most of the transduced cells were found in clusters, suggesting they were clonally derived from the originally transduced cells. Here, 0.2% of dendritic cell fractions were transduced, corresponding to about 2,000 cells per mouse. In addition, macrophage fractions and cells from inguinal and popiloteal lymph nodes of these mice showed some X-gal staining, although the numbers were greatly reduced compared to the dendritic cell fractions. The high density fractions, containing B and T cells, were negative for X-gal staining.

Phenotypes of the transduced cells were further analyzed. B cells, T cells and macrophage fractions were shown to be homogenous following fractionation, as judged by their representative marker profiles for CD45R/B220, Thy-1.2, and F4/80. In contrast, cells adhering to plastic immediately after the cell isolation but not after overnight culturing contained mostly dendritic cells based on several dendritic cell antibody stainings, and are therefore conventionally referred to as dendritic cell fractions, although these fractions may also include some CD45R/B220 positive B cells (about 20%) and F4/80 positive monocytes/macrophages (about 30%). The monocytes/macrophage cells in the “dendritic” cell fraction were likely to be monocytes, since more adherent mature macrophages were separated out by continuous adherence to plastic. Therefore, the percent transduction of genuine dendritic cells and monocytes in the “dendritic cell” fraction was determined. In addition, since some F4/80 positive cells appeared to be transduced at the injection sites, it was determined if those cells traffic to the spleen. Splenocytes from mice transduced with the β-gal gene were fractionated to obtain “dendritic cell” fractions as described above. Cells were sorted by using two color FACS to collect CD45R/B220 positive. F4/80 positive or double negative cells. i.e., B cells, monocytes or dendritic cells, respectively. Each population was then X-gal stained and β-gal activities were found in 0.035% of monocytes and 0.025% of dendritic cells, but not in the B cell fractions. These results show that both splenic dendritic cells and monocytes/macrophages from spleen can be transduced and can express vector-encoded proteins.

Since “dendritic cells” from immunized animals were recognized by immune systems and therefore present antigenic peptides, such cells were studied to determine if they can prime naive spleens in vitro. One group of mice was immunized to express HIV envelope gp160/120. Their splenocytes were fractionated into three groups: B plus T cells; macrophages; and a “dendritic cell” fraction. Naive litter mates were similarly processed. The resulting six different cell groups were mixed to reconstitute splenocyte populations. Irradiated stimulators were added to initiate in vitro CTL cultures. Unfractionated, immunized spleen and splenocytes from mouse immunized with cells expressing HIV envelope gp160/120 were included as controls. The first four combinations, including B plus T cell fractions, from immunized mice were able to induce excellent immune responses. Interestingly, only the “dendritic cell” fractions immunized mice were able to induce immune responses in naive B plus T cell populations, demonstrating that the “dendritic cell” fraction contains antigen presenting cells which can initiate immune responses in vitro. As dendritic cells were previously identified as the only population of cells which can initiate immune responses in vitro, this “dendritic cell” population must include genuine, antigen presenting dendritic cells.

Since the above data show that in vivo transduced dendritic cells and macrophages can induce primary immune responses in vivo and in vitro, the ability of ex vivo transduced cells to stimulate immune responses was also studied. Low density cell fractions were prepared by Percoll gradient separation of naive spleens, followed by stimulation with a cocktail of cytokines to promote cell division. The proliferating cells were transduced with recombinant retroviruses carrying an expression vector encoding HIV gp160/120 using transwell cultures. Aliquots of transduced dendritic and macrophage cells were transferred to naive hosts, followed by in vitro restimulation and CTL analysis. As expected, recipient mice induced excellent CTL responses, confirming the role of these cells in immune induction.

To assess the relative efficacy of transduced dendritic cells to induce immune responses by ex vivo methods, as compared to other cell types, transduced dendritic cells were compared to transduced fibroblasts expressing comparable levels of the same antigen injected by identical injection protocol. On the basis of retrovirus-mediated luciferase expression, transduction efficiency of dendritic cells was approximately 0.1% via identical protocol followed by normalization based on protein content. Based on this transduction efficiency, in the adoptive transfer experiments recipient mice were calculated to have received on average about 5×10² transduced dendritic cells in a total of 5×10⁵ injected cells. The immune response levels induced by these ex vivo transduced dendritic cells are comparable to those generated using the same injection protocol to inject 3×10⁴ to 1×10⁵ fibroblasts expressing, the same antigen. On the basis of this calculation, a single transduced dendritic cell induces an immune response equivalent to that generated by 60 to 200 transduced fibroblast cells.

The data above indicate the relative rarity of protein expressing cells. To estimate the frequency of antigen presenting cells among cells expressing the antigen or protein from which it is derived, the following procedures were used to identify these rare antigen presenting cells at the injection sites and in various lymphoid organs.

Ten C57BL/6 mice were injected with an ovalbumin-encoding recombinant retrovirus. Pooled spleens were fractionated into B, T, macrophage and “dendritic cells”. These potential antigen presenting cells were incubated overnight with B3.Z cells, CTL hybridomas which express the LacZ protein upon antigenic stimulation. These cultures were stained with X-gal to estimate the level of B3.Z activation. As controls, B3.Z cells were incubated with the following reagents: plate-bound anti-T cell receptor antibodies: a known number of E7.G cells transfected with ovalbumin gene: unimmunized splenocytes; or unfractionated immune splenocytes. A standard curve was generated from the frequency of activated blue B3.Z cells incubated with fixed number of E7.G cells and the frequencies of antigen presenting cells in each splenic fraction were estimated therefrom. An average of about 100 antigen presenting cells per spleen was detected in mice injected with total of 10⁷ cfu of the recombinant retrovirus. Pooled splenocytes from these mice yielded 40% lysis in CTL assays conducted at 100:1 effector to target ratios one week after in vitro stimulation.

In another series of experiments, ten C57BL/6 mice were injected with 6×10⁷ cfu of the same gene delivery vehicle as used above. Cells were isolated from the injection sites and lymphoid organs. Comparison to the standard curve generated from recognition of E7.G cells revealed that 1×10⁵ and 4×10⁴ antigen presenting cells were recovered from the inguinal and popiliteal lymph nodes, respectively. 2×10⁴ antigen presenting cells were recovered from the injection sites. This data indicates that antigen presenting cells are quite rare. Accordingly, gene therapy-mediated immune induction can be improved by increasing transduction of selective cell types, as discussed below.

As shown above, a hierarchy of cells impacted by gene delivery vehicle-mediated transduction exists. Cells containing the expression vector can be detected by techniques such as PCR in various sources, including whole organs, purified cell populations, or in situ from tissue sections. Among cells containing the expression vector (Group I), generally only some will express proteins (Group II), although the relative sizes of the Group I and Group II cell populations may be comparable in many instances. Group II cells an be detected by various techniques, including histochemistry and immunohistochemisty. When duration of heterologous protein expression was examined, no loss of expression occurred during the course of the studies above (10 days), although duration of expression is likely to vary depending on the particular gene delivery vehicle employed.

Antigen processing and presentation involve many steps and parameters: processing of antigenic protein into peptides; transportation of peptide to the endoplasmic reticulum; binding of the peptide to appropriate MHC I class molecules; and microenvironments where peptide-MHC class I complexes can interact with cytotoxic T cells. Thus, only subpopulations of protein expressing cells are likely to present antigenic peptide to the immune system (Group III). The results above confirm this hypothesis, and indeed find that antigen presenting cells are quite rare.

Lastly, a distinct group of cells which do not contain the expression vector and therefore can not express the antigen(s), but nonetheless present antigens (Group IV), exist. Examples of these cells include phagocytic cells which engulf primary transduced cells or their fragments and reprocess and present antigenic peptides in the context of their own MHC molecules, i.e., cross-priming.

The results above demonstrate that antigen presentation following direct administration of gene delivery vehicles is mediated by splenic dendritic and macrophage cells. Without being bound to a particular theory, it is likely that the cells which are ultimately transduced at the injection site may have been recruited by the inflammatory reaction caused by needle injury. After transduction, the cells are likely to drain via the lymphatics to the spleen and present antigen. Ex vivo transduced monocytes/macrophages and dendritic cells are thus excellent sources for transferring immune responses. Therefore, autologous or haplotype-matched, transduced dendritic or monocytes/macrophages may be used as in an immunotherapeutic approach to disease treatment or prevention. However, due to logistical and other complications associated with ex vivo gene therapy, directly administering gene delivery vehicles to a patient is preferred. In particularly preferred approaches, in vivo transduction of dendritic cells is performed. To enhance such transduction, patients may be treated by pre-injection or co-injection of gene delivery vehicles encoding cytokines such as GM-CSF to selectively cause proliferation of leukocytes. Alternatively, recombinant versions of such immunomodulatory cofactors may be administered. In an especially preferred approach, gene delivery vehicles are targeted to dendritic cells in vivo. Production of representative examples of such targeted gene delivery vehicles are described below in Example 2.

### Example 2

### Production of Gene Delivery Vehicles Targeted to Dendritic Cells

This example describes production of three representative gene delivery vehicles designed to target dendritic cells in vivo. The first gene delivery vehicle described is a recombinant retrovirus. The second gene delivery described is a recombinant alphavirus, and the third gene deliver, vehicle is a non-viral eukaryotic lathered vector initiation system. These gene delivery vehicles are then purified and combined with a dendritic cell targeting element, after which the preparations are again purified (preferably) and formulated into pharmaceutical compositions.

1. Recombinant Retrovirus Carrying An Expression Vector Encoding A Disease Associated Antigen

A recombinant retrovirus carrying an expression vector encoding a disease associated antigen, namely the cancer associated antigen ras, is described below.

A. Preparation of Retroviral Backbones KT-1 and KT-3B

The Moloney murine leukemia virus (MoMLV) 5′ long terminal repeat (LTR) EcoR I-EcoR I fragment, including gag sequences, from the N2 (Armentano, et al., J. Vir. 61:1647, 1987, Eglitias, et al., Science 230:1395, 1985) vector is ligated into the plasmid SK⁺ (Stratagene, San Diego, Calif.). The resulting construct is designated N2R5. The N2R5 construct is mutated by site-directed in vitro mutagenesis to change the ATG start codon to ATT, preventing gag expression. This mutagenized fragment is 200 base pairs (bp) in length and flanked by Pst I restriction sites. The Pst I-Pst I mutated fragment is purified from the SK⁺ plasmid and inserted into the Pst I site of N2 MoMLV 5′ LTR in plasmid pUC31 to replace the non-mutated 200 bp fragment. The plasmid pUC31 is derived from pUC19 (Stratagene, San Diego, Calif.) in which additional restriction sites Xho I, Bgl II, BssH II and Nco I are inserted between the EcoR I and Sac I sites of the polylinker. This construct is designated pUC31/N2R5gM.

A 1.0 kilobase (Kb) MoMLV 3′ LTR EcoR I-EcoR I fragment from N2 is cloned into plasmid SK⁺ resulting in a construct designated N2R3-. A 1.0 Kb Cla I-Hind III fragment is purified from this construct.

The Cla I-Cla I dominant selectable marker gene fragment from pAFVXM retroviral vector, comprising a SV40 early promoter driving expression of the neomycin (neo) phosphotransferase gene, is cloned into the SK⁺ plasmid. This construct is designated SV⁺ SV₂-neo. A 1.3 Kb Cla I-BstB I gene fragment is purified from the SK⁺ SV₂-neo plasmid.

KT-3B or KT-1 vectors are constructed by a three part ligation in which the Xho I-Cla I fragment containing the gene of interest and the 1.0 Kb MoMLV 3′ LTR Cla I-Hind III fragment are inserted into the Xho I-Hind III site of pUC31/N2R5gM plasmid. This gives a vector designated as having the KT-1 backbone. The 1.3 Kb Cla I-BstB I neo gene fragment from the pAFVXM retroviral vector is then inserted into the Cla I site of this plasmid in the sense orientation to yield a vector designated as having the KT-3B backbone.

B. Preparation of a Recombinant Retrovirus wherein the Expression Vector Carried thereby Codes for a Mutant ras Gene Product

I. Isolation of ras^{*12}

A 700 base pair Hind III fragment containing the entire T24 ras^{*12} coding region is obtained from plasmid HRAS1 (ATCC No. 41000) and ligated into the Hind III site of pSP73 (Promega, Madison, Wis.). This plasmid is designated SP-Val ¹²(100) (see FIG. 1). Plasmids containing ras^{*12} may also be obtained from other sources, such as the American Type Culture Collection (Rockville, Md.).

In order to determine proper orientation of ras^{*12} in pSP73, clones are subjected to Pvu II digestion, and a clone containing a 100 bp digest is selected. This clone is designated SP-Val¹²(100).

E. coli DH5α (Bethesda Research Labs, Gaithersburg, Md.) is transformed with the SP-Val¹² vector construct, and propagated to generate a quantity of plasmid DNA. The plasmid is then isolated and purified, essentially as described by Birnboim et al. (Nuc. Acid Res. 7:1513, 1979; see also, “Molecular Cloning: A Laboratory Manual,” Sambrook et al. (eds.), Cold Spring Harbor Press, p. 1.25 et seq., 1989).

ii. Preparation of a vector containing D ras^{*12}

A Nco I-Sma I fragment from SP-Val¹²(100) is removed by restriction endonuclease cleavage. A Xba I linker (New England Biolabs, Beverly, Mass.) containing a universal stop codon in all three reading frames is inserted 3′ to the ras coding sequence. This process forms a poly Xba I region which can be removed by restriction endonuclease cleavage at Xba I sites followed by ligation. This mutant is designated SP-D-Val¹² and expresses non-activating, truncated ras (ras^{*}) protein.

iii. Insertion of D ras^{*12} into a Retroviral Backbone

N2-ras-neo and N2-ras*-neo retroviral vectors are constructed essentially as described in U.S. Ser. No. 07/586,603. Briefly, this engineered N2 murine recombinant retrovirus contains the SV40 early promoter and the neomycin phosphotransferase gene to facilitate isolation of the infected and transfected cell lines. The N2 Mo MLV gag ATG initiator codon is also altered to ATT by in vitro site-directed mutagenesis in order to increase retroviral titer and enhance the level of expression of transduced genes.

A 350 bp Xho I-Cla I fragment from SP-D-Val¹²(100) is then ligated into the retroviral vector. This construct was designated N2-D-ras^{*}-Val¹².

The full-length SP-Val¹²(100) cDNA is similarly ligated into the retroviral vector to be used as a positive control for transformation. This construct is designated N2-ras-Val¹².

C. Production of Packaging Cell Lines HX and DA

Production of large quantities of recombinant retroviruses can be performed using various retroviral packaging cell lines. In preferred embodiments, packaging cell lines are employed which produce high titers of infectious. RCR-free recombinant retroviruses. Construction of representative examples of such packaging cell lines are described below, as well as in co-owned U.S. Ser. No. 08/367,071, WO 92/05266, U.S. Ser. No. 08/240,030, and U.S. Ser. No. 08/305,699. In particularly preferred embodiments, when the patient to be treated is human, the retroviral component of the GDV is generated from a producer cell made from a human packaging cell line. GDVs so produced are resistant to complement inactivation. Similarly, when the patient to be treated is a non-human mammal, viral particles produced in cells of the same species as the patient are preferred for incorporation into GDVs of the invention.

i. Plasmid DNA Transfection

The following procedure may be used to transiently transfect appropriate packaging cell lines with plasmids encoding retroviral expression vectors according to the invention. For example, the packaging cell line HX (WO 92/05266) is seeded at 5.0×10⁵ cells on a 10 cm tissue culture dish on day 1 with DMEM and 10% FBS. On day 2, the media is replaced with 5.0 mL fresh media 4 hours prior to transfection. A standard calcium phosphate-DNA co-precipitation is performed by mixing 40.0 μL 2.5 M CaCl₂, 10 μg plasmid DNA, and deionized H₂O in a total volume of 400 μL. The DNA-CaCl₂ solution is added dropwise with constant agitation to 400 μl precipitation buffer (50 mM HEPES-NaOH, pH 7.1; 0.25M NaCl and 1.5 mM Na₂HPO₄-NaH₂PO₄). This mixture is incubated at room temperature for 10 minutes. The resultant fine precipitate is added to the HX of cells. The cells are incubated with the DNA precipitate overnight at 37° C. On day 3, the media is aspirated and fresh media is added. The supernatant is removed on day 4, passed through a 0.45 μL filter, and stored at −80° C.

ii. Packaging Cell Line Transduction and Generation of Producer Lines

To increase retroviral titers produced from packaging cells, it is preferable to transduce another packaging cell line with retroviral vectors transiently produced from another cell line. For example, DA (an amphotropic cell line derived from D 17 cells ATCC No. 183, WO 92/05266) cells are seeded at 5.0×10⁵ cells/10 cm tissue culture dish in 10 mL DMEM and 10% FBS, 4 μg/mL polybrene (Sigma, St. Louis, Mo.) on day 1. On day 2, 3.0 mL, 1.0 mL and 0.2 mL of the freshly collected virus-containing HX media is added to the cells. The cells are incubated with the virus overnight at 37° C. In those instances when the retroviral also encodes a selectable marker, e.g., neomycin resistance, on day 3, the media is removed and 1.0 mL DMEM. 10% FBS with 800 μg/mL G418 is added to the plate. Only cells that have been transduced with the vector and expressing the selectable marker will survive. In the case of neomycin resistance, G418 resistant pools can be generated over a period of a week. Typically, a pool of cells is then dilution cloned by removing the cells from the plate and counting the cell suspension, diluting the cells suspension down to 10 cells/mL and adding 0.1 mL to each well (1 cell/well) of a 96 well plate (Corning, Corning, N.Y.). Cells are incubated for 14 days at 37° C. 10% CO₂. As many as twenty-four clones are selected and expanded up to 24 well plates. 6 well plates then 10 cm plates at which time the clones are assayed for expression and the supernatant are collected and assayed for viral titer.

The titer of the individual clones is determined by infection of HT1080 cells, (ATCC No. CCL 121). On day 1, 5.0×10⁵ HT1080 cells are plated on each well of a 6 well microtiter plate in 3.0 mL DMEM, 10% FBS and 4 μg/mL polybrene. On day 2, the supernatant from each clone is serially diluted 10 fold and used to infect the HT1080 cells in 1.0 mL aliquots. The media is replaced with fresh DMEM, 10% FBS media, and the cells incubated with the vector overnight at 37° C. 10% CO₂. On day 3, selection of transduced cells is performed (assuming the presence of a selectable marker in the recombinant vector) by replacing the media with fresh DMEM, 10% FBS media containing the appropriate selection agent, for instance, 800 μg/mL G418 in the case of neomycin resistance. Cells are incubated at 37° C., 10% CO₂ for 14 days at which time G418 resistant colonies are scored at each dilution to determine the viral titer of each clone as cfu/mL.

Using these procedures, cell lines are derived that produce greater than or equal to 1.0×10⁶ cfu/mL in culture. In addition, as those in the art will appreciate, in those instances where selectable markers other than drug resistance, or when no selectable marker is encoded by the recombinant vector, other titer methods, such as antibody-based assays, PCR assays, etc., may be employed.

The packaging cell line HX is transduced with vector generated from the DA vector producing cell line in the same manner as described for transduction of the DA cells from HX supernatant.

Transduction of the DA or HX cells with vectors lacking a neo selectable marker was performed as described above. However, instead of adding G418 to the cells on day S3, the cells are cloned by limiting dilution. Titer and RCR assays are performed as described in WO 92/05266 and co-owned U.S. Ser. No. 08/367,071.

D. Transformation (Tumorigenicity) Assay

Rat 2 cells (ATCC No. CRL 1764) are grown in Dulbecco-Vogt modified Eagle medium supplemented with 10% fetal bovine serum. Rat 2 cells are plated at 10⁶ cells per 5 cm dish 1 day before transfection. The cells are transfected with 0.1-1.0 μg of construct DNA as previously described (Graham and Van Der Eb. 1973; Corsaro and Pearson, 1981). The next day the cells are trypsinized and seeded into three 5 cm dishes and fed every three days thereafter with medium containing 5% fetal bovine serum plus 2 ×10⁻⁶M dexamethasone (this enhances the contrast between transformed and non-transformed rat 2 cell morphology). Transformed foci are visible after about 1 week. The plates are stained and foci counted after about three weeks (Miller et al., Cell 36:51, 1984).

Cells transfected with ras^{*} recombinant retroviruses formed transformed foci, whereas those transfected with D ras^{*12} recombinant retroviruses did not.

E. Cytotoxicity Assay

Six- to eight-week-old female BALB/c mice (Harlan Sprague-Dawley, Indianapolis, Ind.) are injected once intraperitoneally (i.p.) with 5×10⁶ irradiated (10,000 rads, 60#C) vector transfected cells (e.g., BC-ras^{*}). Animals are sacrificed 7 days later and the splenocytes (3×10⁶/mL) cultured in vitro with irradiated syngeneic transduced cells (6×10⁴/mL) in flasks (T-25, Corning, Corning, N.Y.). Culture medium consists of RPMI 1640, heat-inactivated fetal bovine serum (5%, Hyclone, Logan, Utah), sodium pyruvate (1 mM), gentamicin (50 μg/ml) and 2-mercaptoethanol (10⁻⁵M, Sigma Chemical, St. Louis, Mo.). Effector cells are harvested 4-7 days later and tested using various Effector:Target cell ratios in 96 well microtiter plates (Corning, Corning, N.Y.) in a standard 4-6 hour assay. The assay employs Na₂⁵¹CrO₄-labeled (Amersham, Arlington Heights, Ill.) (100 uCi, 1 hr at 37#C) target cells (1×10⁴ cells/well) in a final volume of 200 ul. Following incubation, 100 ul of culture medium is removed and analyzed in a Beckman gamma spectrometer. Spontaneous release (SR) is determined as CPM from targets plus medium and maximum release (MR) is determined as CPM from targets plus 1M HCl. Percent target cell lysis is calculated as: [(Effector cell+target CPM)−(SR)/(MR)−(SR)]×100. Spontaneous release values of targets are typically 10%-20% of the MR.

2. Recombinant Alphavirus Carrying An Expression Vector Encoding A Disease Associated Antigen

A recombinant alphavirus carrying an expression vector encoding a disease associated antigen, namely the HBV e/core antigen, is described below.

Construction of an alphaviral vector backbone derived from Sindbis virus, designated pKSSINBV, is described in WO 95/07994 and co-owned U.S. Ser. No. 08/404,796. The HBV e/core sequence is isolated and cloned into this vector as follows:

A 1.8 Kb fragment containing the entire precore/core coding region of hepatitis B is obtained from plasmid pAM6 (ATCC No 45020) following Bam HI digestion and gel purification, and ligated into the Bam HI site of KS II+ (Stratagene, La Jolla, Calif.). This plasmid is designated KS II+ HBpc/c. Xho I linkers are added to the Stu I site of precore/core in KS II+ HBpc/c (at nucleotide sequence 1,704), followed by cleavage with Hinc II (at nucleotide sequence 2.592). The resulting 877 base pair Xho I-Hinc II precore/core fragment is cloned into the Xho I/Hinc II site of SK II+. This plasmid is designated SK⁺HBe.

The precore/core gene in plasmid KS II+ HB pc/c was sequenced to determine if the precore/core coding region was correct. This sequence was found to have a single base-pair deletion which causes a frame shift at codon 79 that results in two consecutive in-frame TAG stop codons at codons 84 and 85. This deletion was corrected by PCR overlap extension (Ho et al., Gene 77:51, 1989) of the precore/core coding region in plasmid SK⁺ HBe. Four oligonucleotide primers of appropriate size and nucleotide sequence were used for the three PCR reactions performed to correct the deletion. See WO 95/07994 for additional details.

The first PCR reaction corrected the deletion in the antisense strand and the second reaction corrected the deletion in the sense strand. Specifically, PCR reactions one and two corrected the mutation from CC to CCA which occurs in codon 79 and a base pair substitution from TCA to TCT in codon 81. Primer 1 contained two consecutive Xho I sites 10 bp upstream of the ATG codon of HBV e coding region and primer 4 contained a Cla I site 135 bp downstream of the stop codon of HBV precore/core coding region. The products of the first and second PCR reactions were extended in a third PCR reaction to generate one complete HBV precore/core coding region with the correct sequence.

The desired PCR reaction product was purified by 1.5% agarose gel electrophoresis and transferred onto NA 45 paper (Schleicher and Schuell, Keene, N.H.). The desired 787 bp DNA fragment was eluted from the NA 45 paper by incubating for 30 minutes at 65° C. in 400 μl high salt buffer (1.5M NaCl, 20 nM Tris, pH 8.0, and 0.1 nM EDTA). Following elution, 500 μl of phenol:chloroform:isoamyl alcohol (25:24:1) was added to the solution. The mixture was vortexed and then centrifuged 14,000 rpm for 5 minutes in a Brinkmann Eppendorf centrifuge (5415L). The aqueous phase, containing the desired DNA fragment, was transferred to a fresh 1.5 mL microfuge tube and 1.0 mL of 100% EtOH is added. This solution is incubated on dry ice for 5 minutes, and then centrifuged for 20 minutes at 10,000 rpm. The supernatant was decanted, and the pellet rinsed with 500 l of 70% EtOH. The pellet was dried by centrifugation at 10,000 rpm under vacuum, in a Savant Speed-Vac concentrator, and then resuspended in 10 μL of deionized H₂O. One microliter of the PCR product was analyzed by 1.5% agarose gel electrophoresis. The 787 Xho I-Cla I precore/core PCR amplified fragment was cloned into the Xho I-Cla I site of SK⁺ plasmid. This plasmid is designated SK⁺HBe-c. E. coli DH5α (Bethesda Research Labs, Gaithersburg, Md.) is transformed with the SK⁺HBe-c plasmid and propagated to generate plasmid DNA. The plasmid is then isolated and purified, essentially as described by Birnboim et al. (Nuc. Acid Res. 7:1513, 1979: see also Molecular Cloning: A Laboratory Manual, Sambrook et al. (eds.). Cold Spring Harbor Press, 1989). The SK⁺HBe-c plasmid was analyzed to confirm the sequence of the precore/core gene.

Construction of a Sindbis vector expressing the HBVe sequence was accomplished by digesting the SK⁺HB e-c plasmid with Xho I and Xba I to release the cDNA fragment encoding HBVe-c sequence. The fragment was then isolated by agarose gel electrophoresis, purified by GENECLEAN, and inserted into pKSSINBV (see WO 95/07994). prepared by digestion with Xho I and Xba I, and treated with CIAP. This vector was designated pKSSIN-HBe.

The above Sindbis HBV expression vector may also be modified to coexpress other gene products, for instance, an immunomodulatory cofactor, a selectable marker, and/or one or more additional disease associated antigens. Such alphavirus expression vectors can be packaged into infectious viral particles by following procedures described in detail in WO 95/07994 and co-owned U.S. Ser. No. 08/404,796.

3. Purification of Recombinant Viruses

Recombinant viruses prepared for use in accordance with the invention are preferably purified prior to formulation or association with one or more dendritic cell targeting elements. Particularly preferred procedures for purifying recombinant lipid enveloped viruses are provided in U.S. Pat. No. 5,447,859.

4. A Eukaryotic Layered Vector Initiation System Encoding A Disease Associated Antigen

A non-viral eukaryotic layered vector initiation system (ELVIS) encoding a disease associated antigen, namely the HBV e/core antigen, is briefly described below. The SK⁺HB e-c plasmid is digested with Xho I and Not I to release the cDNA fragment encoding HBVe-c sequence. The fragment is then isolated by agarose gel electrophoresis, purified using GENECLEAN™, and inserted into pVGELVIS-SINBV-linker vector (see WO 95/07994 and co-owned 08/404,796), previously prepared by digestion with Xho I and Not I. This construct is designated pVGELVIS-HBe. If desired, this construct can be purified prior to use or combination with a dendritic cell targeting element and/or other compounds, such as lipids and polycations.

5. Addition of Dendritic Cell Targeting Elements

Techniques for adding targeting elements, for instance, dendritic cell targeting elements, to gene delivery vehicles are provided in co-owned U.S. Ser. No. 08/440,616, U.S. Ser. No. 08/577,282 and U.S. Ser. No. 08/580,541.

6. Formulation of Gene Delivery Vehicles for Targeted Transduction of Dendritic Cells

Recombinant viruses made in accordance with the teaching provided herein may be formulated by into various pharmaceutical compositions. Representative formulations useful in the practice of the present invention are described in WO 95/10601 and co-owned U.S. Ser. No. 08/153,342. Many formulations can also be developed for non-viral gene delivery systems. Representative examples of such formulations are provided in WO 90/11092 and WO 95/07994.

### Example 3

### Murine Dendritic Cells Transduced with Retroviral Vector to Induce Prophylactic and Therapeutic Immune Responses Against Tumor Specific Antigens

1. Tumor Therapy

Human papillomavirus (HPV) has been implicated in the genesis of several human cancers, particularly squamous tumors of the cervix and anogenital region. The E7 proteins from some papilloma virus types mediate oncogenic transformation of cervical epithelial cells by binding to the tumor suppressor gene Rb product. The E7 gene is immortalizing and can transform both primary rat cells (Kanda, T. et al., J. Virol. 62:610-613, 1988) and primary human fibroblasts (Watanabe, S. et al. J. Virol, 63, 965-969, 1989). Mutational analysis of E7 aimed at elucidating the relationship between structure and function of the protein has revealed that a single point mutation at amino acid 24 removed the transforming ability while the transactivating activity was retained (Edmonds, C. et al., J. Virology 63, 2650-2656, 1989) and hence this E7 variant was named p24. Theoretically, E7 protein which is present in the majority of cervical cancers is an attractive target for an in vivo stimulated T-cell response.

Since murine cell lines are non-permissive to HPV infections, two surrogate HPV regions were generated by transducing retroviral vectors from the DA/p24 producer line. The DA/p24 producer line was constructed as described in Example 2 above, except that the p24 gene (Edmonds, C. et al., J. Virology 63, 2650-2656, 1989) was inserted into the retroviral backbone instead of the ras gene. Mammary carcinoma cell line, JC (ATCC#CRL-2116), and colorectal carcinoma cell line, CT26 (Michael Brittain at Baylor College of Medicine) were transduced with DA/p24 to generate JC.p24 and CT26.p24 respectively.

Transduced dendritic cell fractions were prepared as follows. Naive mice were sacrificed and the splenic dendritic cell (SP-DC) fraction was prepared as described in Example 1. Bone marrow dendritic cells (BM-DC) were prepared by flushing the longbones followed by complement mediated elimination of B cells. They were put into RPMI 1640 media containing GN4-CSF (500U/ml) and IL-4 (1000U/ml). DA/p24 was seeded in transwells (Corning Costar, Cambridge, Mass.) with a pore size of 0.45 micron, which does not allow any leakage of producer cells across the membrane. The dendritic cell fraction was added to low adherence tissue culture wells (Corning Costar, Cambridge, Mass.) and transwells containing DA/p24 were placed above the dendritic cell fraction. After seven days of co-cultivation, the recovered cells were washed three times with PBS before injection into naive recipient mice for prophylaxis or tumor bearing mice for therapy experiments. D2SC/1, a parental immortalized dendritic cell line (WO 94/28113) was transduced with either beta-gal or p24 antigens. The resultant two immortalized dendritic cell lines D2SC/1.beta-gal and D2SC/1.p24. were used as controls.

For therapy experiments, either JC.p24 or CT26.p24 tumor bearing BALB/c mice were treated with syngenic BM-DC or SP-DC dendritic cells transduced by co-cultivation with DA/p24 producer lines. The tumors were inoculated on day 1 followed by weekly treatment with p24 transduced BM-DC or SP-DC. For immunization experiments, naive BALB/c mice were immunized twice with p24 transduced BM-DC or SP-DC on days 1 and 7, followed by JC.p24 or CT26.p24 challenges on day 14. All mice were measured for tumor volume twice a week. For all experiments, the mean tumor volumes were plotted against days following tumor inoculation.

A. C726.p24 Model

2×10⁵ CT26.p24 cells were injected into 50 BALB/c mice (5 groups of 10 mice each) and treated with p24 transduced BM or spleen derived dendritic cells on days 7, 14 and 21. D2SC/1.beta-gal and D2SC/1.p24 were injected at 5×10⁶ cells per mouse per therapy. BM-DC and SP-DC were injected at 2-3×10⁶ cells per mouse or 4-10×10⁵ cells per mouse respectively, per therapy. Efficiencies of BM-DC and SP-DC were estimated to be 0.1-1% and therefore 2×10³ to 3×10⁴ (BM-DC) or 4×10² to 1×10⁴ (SP-DC) transduced dendritic cells were believed to be injected per mice per therapy.

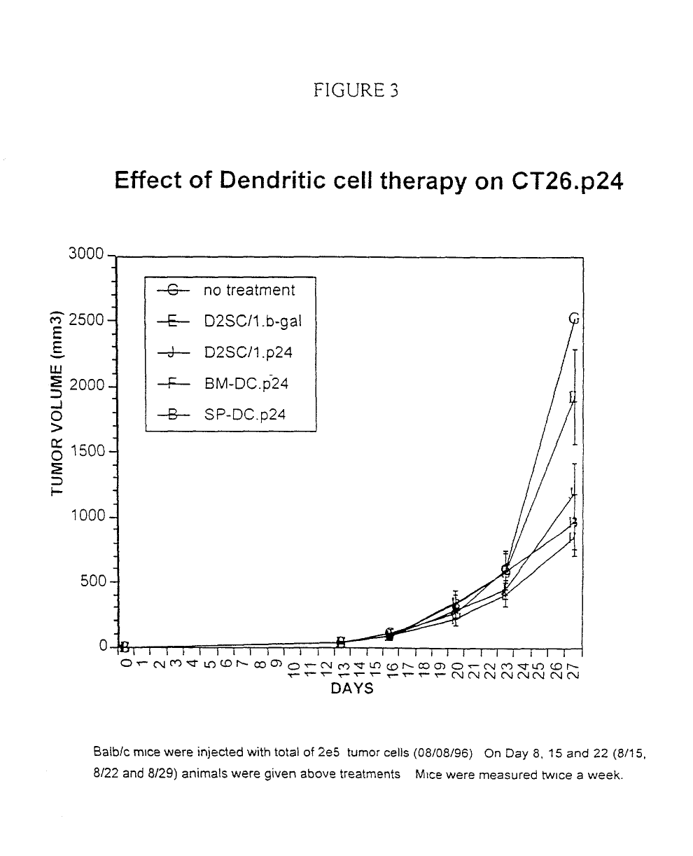

The results for the effects of dendritic cell therapy using CT26.p24 cells are shown in FIG. 3. Mice immunized with p24 transduced SP-DC or BM-DC, showed less than a 30% decrease in tumor volume when compared to control mice.

B. JC.P24 Model

3×10⁵ JC.p24 cells were injected into 50 BALB/c mice (5 groups of 10 mice each) and treated with transduced dendritic cells on days 7, 14 and 21. D2SC/1.beta-gal and D2SC/1.p24 were injected at 5×10⁶ cells per mouse per therapy. BM-DC and SP-DC were injected at 2-3e6 cells per mouse or 4-10×10⁵ cells per mouse respectively, per therapy. Transduction efficiency of BM-DC and SP-DC by transwell co-cult was estimated to be 0.1-1% and therefore 2×10³ to 3×10⁴ (BM-DC) or 4×10² to 1×10⁴ (SP-DC) transduced dendritic cells were injected per mice per immunization.

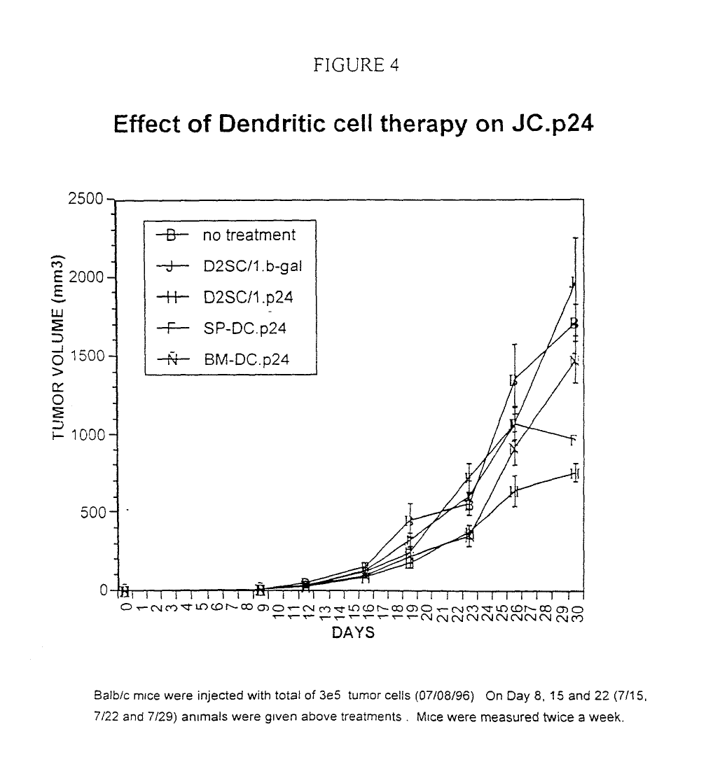

The results for the effects of dendritic cell therapy using JC.p24 are demonstrated in FIG. 4. SP-DC immunized mice displayed approximately a 60% decrease in tumor volume as compared to control animals. Despite the fact that BM-DC vaccinated animals were injected with BM-DC only once, some protection was still observed. Several animals showed reduction of tumor burdens. D2SC/1.beta-gal immunized mice displayed intermediate levels of protection between unimmunized controls and D2SC/1.p24 immunized animals.

2. Tumor Prophylaxis

A. JC.p24 Tumor

50 BALB/c mice were immunized twice, on day 1 and 7. 3×10⁵ JC.p24 cells were injected into 50 BALB/c mice (5 groups of 10 mice each)) and treated with transduced dendritic cells on days 7, 14 and 21. D2SC/1.beta-gal and D2SC/1.p24 were injected at 5×10⁶ cells per mouse per therapy. BM-DC and SP-DC were injected at 2-3×10⁶ cells per mouse or 4-10×10⁵ cells per mouse respectively, per therapy. Transduction efficiency of BM-DC and SP-DC by transwell co-cult was estimated to be 0.1-1% and therefore 2×10³ to 3×10⁴ (BM-DC) or 4×10² to 1×10⁴ (SP-DC) transduced dendritic cells were injected per mice per immunization.

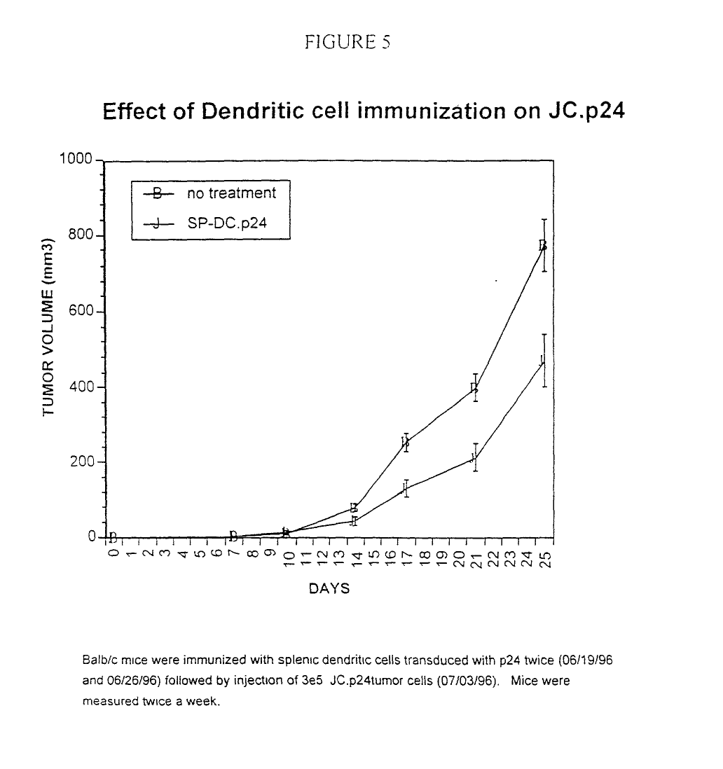

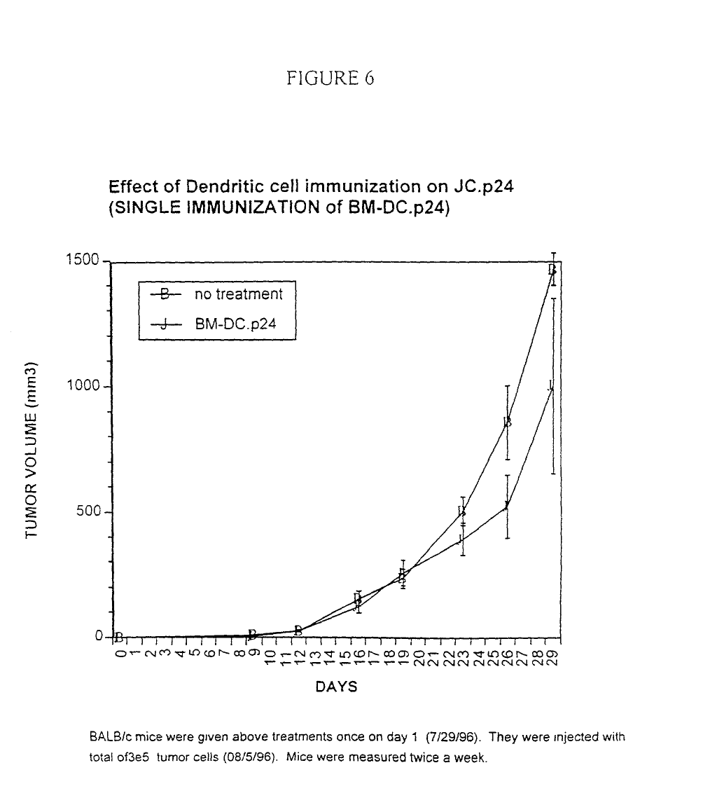

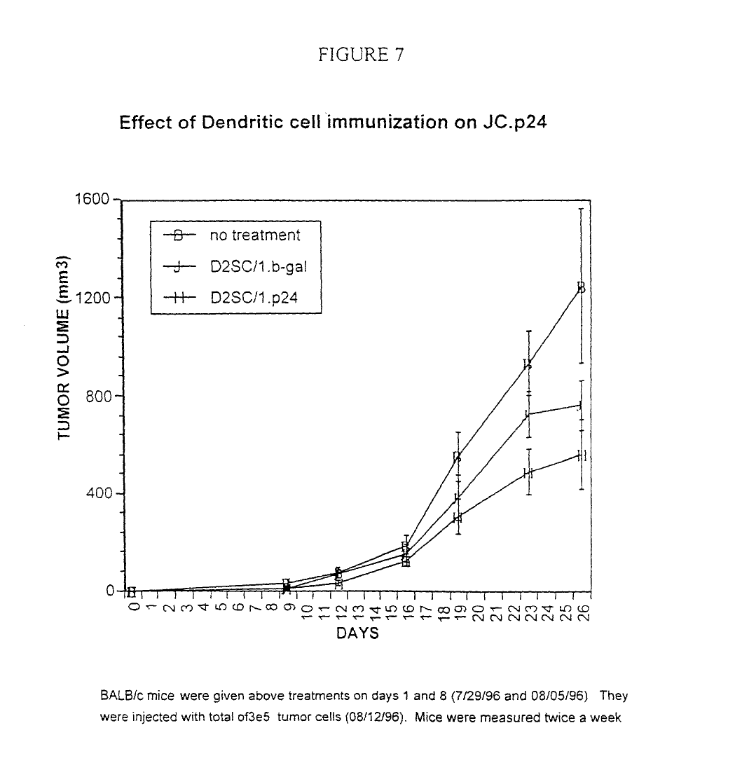

The results for the effects of dendritic cell immunization using JC.p24 are displayed in FIG. 5. Despite the fact that BM-DC vaccinated animals were injected only once with BM-DC, some protection was still observed (FIG. 6). Several animals showed reduction of tumor burdens. D2SC/1.beta-gal immunized mice (FIG. 7) showed intermediate levels of protection between unimmunized controls and D2SC/1.p24 immunized animals.

B. C726.p24 Model

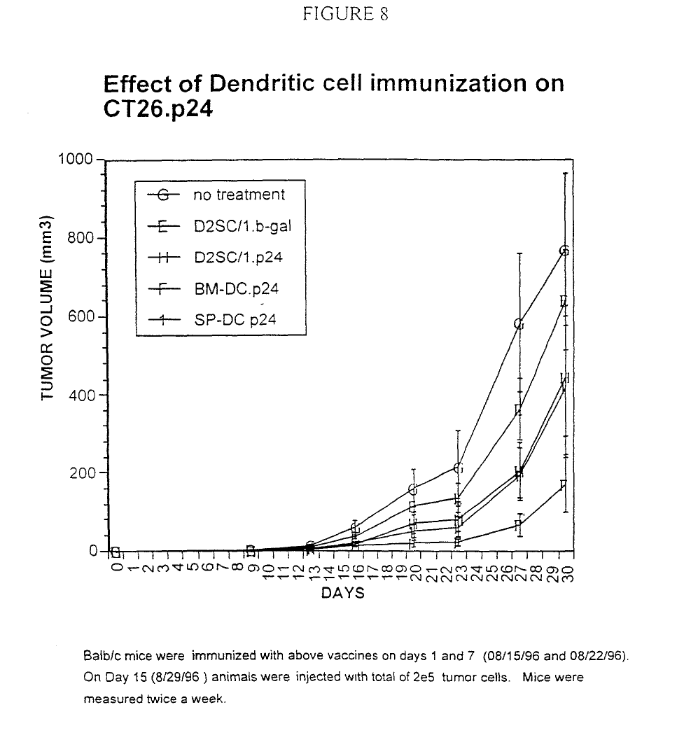

It has been suggested in the literature (Zitvogel, et al., Exp J. Med. 183:87, 1996) that dendritic cell based prophylaxis and therapy is more efficacious if the tumor is immunogenic. Dendritic cell prophylaxis or therapy can achieve complete protection or regression of immunogenic tumor whereas only partial protection or regression of weakly immunogenic tumors can be achieved. Since JC.p24 is weakly immunogenic and therefore presumably less responsive to immunotherapy, similar prophylaxis experiments using immunogenic CT26.p24 displayed better protection (see FIG. 8).

3. Comparison to Other Methods of Dendritic Cell Loading

The following experiment showed that retroviral transduction was equivalent to protein or peptide loading of dendritic cells in inducing immune responses. Beta-gal was used as a surrogate tumor antigen in CT26.beta-gal, CT26 tumors transduced with DA/beta-gal vector. Immune responses were induced by using bone marrow dendritic cells either transduced with beta-gal or loaded with antigenic peptide or protein. The following groups were compared for immune induction and the results showed that the CTL response elicited from all these groups was comparable. This result was significant since dendritic cell loading of peptide or protein were considered to be the best way to elicit immune responses. Similar efficacy was achieved with the use of optimized transduction methods.

1. No treatment

2. BM-DC.beta-gal (1×10⁶ per mouse) transduction (DA/beta-gal co-cultivation, 7 days)

3. Protein o/n incubation (100 ug/ml)

4. Peptide loading (20 ng/ml)+hb2m (10 ug/ml) for 2 hours (human beta-2 microglobulin is a standard protocol for cell loading of antigenic peptide)

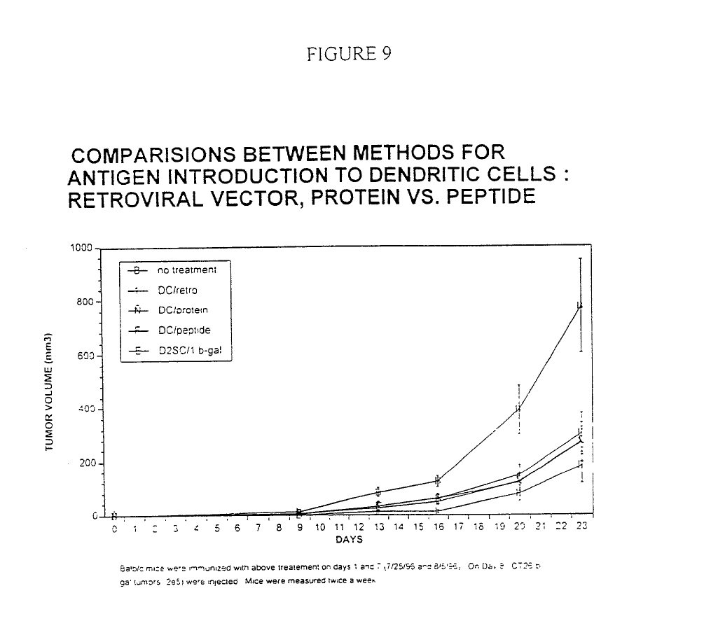

FIG. 9 shows the comparison between methods for antigen introduction to dendritic cells. As shown in this figure, equivalent levels of protection for tumor challenges were observed from animals immunized with retroviral vector transduced dendritic cells and dendritic cells loaded with antigenic peptide or protein. Since the molar ratio of beta-gal peptide or protein used to load dendritic cells was in vast excess (>1 million fold), while the retroviral vector transduction efficiency was estimated to be only 1%, it was highly encouraging that the retroviral transduction achieved the equivalent protection to peptide or protein loading.

The following table shows that the level of protein expression required for immune induction by dendritic cell is only 1% of that by fibroblast. Specifically, net CTL lysis, equivalent to fibroblast immunized animals, was observed in mice immunized with transduced dendritic cells with 1/100 of the protein expression. This results demonstrated the superior efficiency of antigen presentation by dendritic cells.

| | Relative | Mean tumor | |
|---|---|---|---|
| | amounts of | volumes | Net CTL % |
| | beta-gal per | (mm³) on day | lysis at |
| Groups | immunization | 23 | E:T = 100:1 |
| No treatment | 0 | 773 ± 173 | 0 |
| Beta-gal transduced | 1 | 274 ± 50 | 30 |
| dendritic cells | | | |
| D2SC.1/beta-gal | 5 | 182 ± 64 | 95 |
| immortalized | | | |
| dendritic cells | | | |
| BC/beta-gal | 100 | not available | 51 |

While the present invention has been described above both generally and in terms of preferred embodiments, it is understood that variations and modifications will occur to those skilled in the art in light of the description, supra. Therefore, it is intended that the appended claims cover all such variations coming within the scope of the invention as claimed.

Additionally, the publications, patents, patent applications, and other materials cited to illuminate the background of the invention, and in particular, to provide additional details concerning its practice as described in the detailed description and examples, are hereby incorporated by reference in their entirety.

We claim:

1. A gene delivery vehicle comprising a dendritic cell targeting element and an expression vector which directs expression of at least one disease associated antigen.

2. A gene delivery vehicle according to claim 1, wherein the expression vector is carried by a recombinant virus.

3. A gene delivery vehicle according to claim 2, wherein the recombinant virus is a recombinant retrovirus.

4. A gene delivery vehicle according to claim 2, wherein the recombinant virus is an alphavirus.

5. A gene delivery vehicle according to claim 1, wherein the dendritic cell targeting element is selected from the group consisting of a high affinity binding pair, an antibody reactive against a dendritic cell surface marker, and an antigen binding domain derived from an antibody reactive against a dendritic cell surface marker.

6. A gene delivery vehicle according to claim 5, wherein the dendritic cell targeting element is a high affinity binding pair selected from the group consisting of biotin/avidin, cytostatin/papain, and phosphonate/carboxypeptidase A.

7. A gene delivery vehicle according to claim 5, wherein the dendritic cell targeting element is an antibody reactive against a dendritic cell surface marker selected from the group consisting of CD 11c, CD 54, CD 58, CD 25, CD 11a, CD 23, CD 32, CD 40, CD 1, CD 45, MHC Class I, MHC Class II, Mac-1, Mac-2, and Mac-3.

8. A gene delivery vehicle according to claim 5, wherein the dendritic cell targeting element is an antigen binding domain derived from an antibody reactive against a dendritic cell surface marker selected from the group consisting of CD 11c, CD 54, CD 58, CD 25, CD 11a, CD 23, CD 32, CD 40, CD 1, CD 45, MHC Class I, and MHC Class II.

9. A gene delivery vehicle according to claim 2, wherein the dendritic cell targeting element is a hybrid envelope protein of the recombinant virus.

10. A gene delivery vehicle according to claim 1, wherein the expression vector directs expression of an antigen associated with a disease selected from the group consisting of cancer, heart disease, a bacterial infection, a parasitic infection, and a viral infection.

11. A gene delivery vehicle according to claim 10, wherein the expression vector also directs expression of an immunomodulatory cofactor.

12. A pharmaceutical composition comprising gene delivery vehicles according to claim 1 and a pharmaceutically acceptable carrier.

13. An in vivo method of producing a genetically modified dendritic cell, the method comprising administering to an animal a gene delivery vehicle targeted to a dendritic cell, wherein the gene delivery vehicle comprises a dendritic cell targeting element and an expression vector which directs expression of at least one disease associated antigen.

14. A method according to claim 13, wherein the gene delivery vehicle is a recombinant virus.

15. A gene delivery vehicle according to claim 13, wherein the dendritic cell targeting element is selected from the group consisting of a high affinity binding pair, an antibody reactive against a dendritic cell surface marker, and an antigen binding domain derived from an antibody reactive against a dendritic cell surface marker.

16. A gene delivery vehicle according to claim 14, wherein the dendritic cell targeting element is a hybrid envelope protein of the recombinant virus.

17. The method of claim 13, wherein said gene delivery vehicle is administered to said dendritic cells ex vivo.

18. A method of stimulating a prophylactic immune response in an animal, the method comprising administering to the animal a prophylactically effective amount of a gene delivery vehicle comprising a dendritic cell targeting element and an expression vector which directs expression of at least one disease associated antigen.

19. A method according to claim 18, wherein the gene delivery vehicle is a recombinant virus.

20. A method according to claim 18, wherein the dendritic cell targeting element is selected from the group consisting of a high affinity binding pair, an antibody reactive against a dendritic cell surface marker, and an antigen binding domain derived from an antibody reactive against a dendritic cell surface marker.
